(19) United States
(12) Patent Application Publication
(10) Pub. No.: US 2026/0094716 A1
(43) Pub. Date: Apr. 2, 2026

(54) BIOMARKER FOR TRANSPLANTATION TOLERANCE INDUCED BY APOPTOTIC DONOR LEUKOCYTES

(71) Applicant: Regents of the University of Minnesota, Minneapolis, MN (US)

(72) Inventors: Amar Singh, Minneapolis, MN (US); Bernhard J. Hering, Minneapolis, MN (US); Sabarinathan Ramachandran, Minneapolis, MN (US)

(73) Assignee: Regents of the University of Minnesota, Minneapolis, MN (US)

(21) Appl. No.: 19/342,280

(22) Filed: Sep. 26, 2025

Related U.S. Application Data
(60) Provisional application No. 63/699,947, filed on Sep. 27, 2024.

(51) Int. Cl. G16H 50/20; G01N 33/50

(52) U.S. Cl. CPC G16H 50/20; G01N 33/505; G01N 2800/52

(57) ABSTRACT

In certain embodiments, the present invention provides methods of identifying and treating a transplant recipient patient having transplantation tolerance induced by apoptotic donor leukocytes infused under cover of transient immunotherapy.

### CROSS-REFERENCE TO RELATED APPLICATION

This application claims priority to U.S. Provisional Application No. 63/699,947 that was filed on Sep. 27, 2024. The entire content of the applications referenced above is hereby incorporated by reference herein.

### STATEMENT REGARDING FEDERALLY SPONSORED RESEARCH

This invention was made with government support under AI166163 and AI102463 awarded by the National Institutes of Health. The government has certain rights in the invention.

### BACKGROUND OF THE INVENTION

For many patients with end-stage organ failure, a transplant has become the most effective treatment option. Current immunosuppressive regimens effectively prevent acute rejection; however, their significant morbidity and their lack of efficacy in preventing chronic rejection remain serious problems. A growing population of chronically immunosuppressed transplant recipients continue to struggle with such problems, which adversely affect their survival. Inducing tolerance to allografts would remove the need for maintenance immunotherapy and improve long-term allograft survival; yet, despite its first demonstration in small animal models more than 65 years ago and its clinical significance, tolerance has been achieved in only a very few patients through mixed hematopoietic chimerism, which requires extensive conditioning therapy. Likewise, in translational models in monkeys, only mixed chimerism has nearly consistently induced tolerance to same-donor kidney allografts.

In nonhuman primate studies, an apoptotic donor leukocyte regimen was consistently effective and required much less intense, short-term immunotherapy. Because of its efficacy and its very favorable safety profile, this regimen is the first clinically translatable, nonchimeric transplantation tolerance regimen. A biomarker for monitoring the induction, maintenance, and loss of transplant tolerance in human recipients is required.

### SUMMARY OF THE INVENTION

This present invention identifies a biomarker for monitoring the consolidation, maintenance, and loss of tolerance in human recipients of solid organ, tissue and cellular allotransplants.

A method of identifying a transplant recipient patient having transplantation tolerance induced by donor antigen administered under cover of transient immunotherapy, comprising: (a) assaying a first blood sample from the patient to detect a baseline frequency of target cells, wherein the first blood sample is obtained pre-tolerization, pre-transplant, and pre-initiation of transient immunotherapy, (b) assaying a second blood sample from the patient to detect a post-procedure frequency of target cells, wherein the second sample is obtained post-tolerization, post-transplant, and post-initiation of transient immunotherapy; and (c) identifying the patient as having transplantation tolerance/immune acceptance induced by the donor antigen when the post-procedure frequency is at least 1.5-fold greater than the baseline frequency, wherein the target cells are T regulatory (Treg) cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻FOXP3⁺, and/or wherein the target cells are T regulatory Type 1 (Tr1) cells having one or more combination of markers PD-1^{hi}CD69^{hi}Helios^{hi}, TIGIT^{hi} CCR2^{hi}ST2^{hi}GrnzB⁺FOXP3⁻, Areg^{hi} TIGIT^{hi} CCR2^{hi} GrnzB⁺ ST2^{hi} FOXP3⁻ and/or CD4⁺CD49b⁺LAG3⁺ and/or wherein the target cells are exhaust CD4 T cells (Tex) having markers PD-1⁺ EOMES⁺ CD127^{hi} Helios⁺TOX⁺ TCF-1⁻.

In certain embodiments, the post-procedure frequency is at least 1.5-fold greater than the baseline frequency. In certain embodiments, the frequency is at least 1.5, 2, 3, 4, 5, 6, 7, 8, 9, 10, 20, 30, 40, 50, 60, 70, 80, 90, or 100-fold greater than the baseline frequency.

In certain embodiments, the transplant recipient patient has transplantation tolerance maintained. In certain embodiments, the transplant recipient patient had immune tolerance induced but failed. In certain embodiments, immune tolerance was not induced in the transplant recipient patient.

In certain embodiments, the present invention provides a method, comprising: (a) obtaining a first blood sample from a transplant recipient patient to detect a baseline frequency of target cells, wherein the first blood sample is obtained pre-tolerization, pre-transplant, and pre-initiation of transient immunotherapy, (b) obtaining a second blood sample from the patient to detect a post-procedure frequency of target cells, wherein the second sample is obtained post-tolerization, post-transplant, and post-initiation of transient immunotherapy, (c) assaying the first and second blood samples to detect levels of target cells before and after tolerization, (d) identifying the transplant recipient patient as having transplantation tolerance/immune acceptance induced by donor antigens infused under cover of transient immunotherapy when the post-procedure frequency is at least 1.5-fold greater than the baseline frequency, wherein the target cells are T regulatory (Treg) cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻FOXP3⁺, and/or wherein the target cells are T regulatory Type 1 (Tr1) cells having one or more combination of markers PD-1^{hi}CD69^{hi}Helios^{hi}, TIGIT^{hi} CCR2^{hi}ST2^{hi}GrnzB⁺FOXP3⁻, Areg^{hi} TIGIT^{hi} CCR2^{hi} GrnzB⁺ ST2^{hi} FOXP3⁻ and/or CD4⁺CD49b⁺LAG3⁺ and/or wherein the target cells are exhaust CD4 T cells (Tex) having markers PD-1⁺EOMES⁺ CD127^{hi} Helios⁺TOX⁺ TCF-1⁻.

In certain embodiments, the post-procedure frequency is at least 1.5-fold greater than the baseline frequency. In certain embodiments, the frequency is at least 1.5, 2, 3, 4, 5, 6, 7, 8, 9, 10, 20, 30, 40, 50, 60, 70, 80, 90, or 100-fold greater than the baseline frequency.

In certain embodiments, the present invention provides a method of identifying a transplant recipient patient having transplantation tolerance induced by donor antigen administered under cover of transient immunotherapy, comprising:
(a) assaying a first blood sample from the patient to detect a baseline frequency of target cells, wherein the first blood sample is obtained pre-tolerization, pre-transplant, and pre-initiation of transient immunotherapy,
(b) assaying a second blood sample from the patient to detect a post-procedure frequency of target cells, wherein the second sample is obtained post-tolerization, post-transplant, and post-initiation of transient immunotherapy; and
(c) identifying the patient as having transplantation tolerance/immune acceptance induced by the donor antigen when the post-procedure frequency is at least 1.5-fold greater than the baseline frequency,
wherein the target cells are T regulatory (Treg) cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻ FOXP3⁺; and/or
wherein the target cells are T regulatory Type 1 (Tr1) cells having one or more combination of markers PD-1^{hi}CD69^{hi}Helios^{hi}, TIGIT^{hi} CCR2^{hi}ST2^{hi}GrnzB⁺FOXP3⁻, Areg^{hi} TIGIT^{hi} CCR2^{hi} GrnzB⁺ ST2^{hi} FOXP3⁻ and/or CD4⁺CD49b⁺LAG3⁺; and/or
wherein the target cells are exhaust CD4 T cells (Tex) having markers PD-1⁺EOMES⁺ CD127^{hi} Helios⁺TOX⁺ TCF-1⁻.

In certain embodiments, the present invention provides a method, comprising:
(a) obtaining a first blood sample from a transplant recipient patient to detect a baseline frequency of target cells, wherein the first blood sample is obtained pre-tolerization, pre-transplant, and pre-initiation of transient immunotherapy,
(b) obtaining a second blood sample from the patient to detect a post-procedure frequency of target cells, wherein the second sample is obtained post-tolerization, post-transplant, and post-initiation of transient immunotherapy,
(c) assaying the first and second blood samples to detect levels of target cells before and after tolerization, and
(d) identifying the transplant recipient patient as having transplantation tolerance/immune acceptance induced by donor antigens infused under cover of transient immunotherapy when the post-procedure frequency is at least 1.5-fold greater than the baseline frequency,
wherein the target cells are T regulatory (Treg) cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻ FOXP3⁺; and/or wherein the target cells are T regulatory Type 1 (Tr1) cells having one or more combination of markers PD-1^{hi}CD69^{hi}Helios^{hi}, TIGIT^{hi} CCR2^{hi}ST2^{hi}GrnzB⁺FOXP3⁻, Areg^{hi} TIGIT^{hi} CCR2^{hi} GrnzB⁺ ST2^{hi} FOXP3⁻ and/or CD4⁺CD49b⁺LAG3⁺; and/or wherein the target cells are exhaust CD4 T cells (Tex) having markers PD-1⁺ EOMES⁺ CD127^{hi} Helios⁺TOX⁺ TCF-1⁻;
wherein the identification is with a multiparameter single flowcytometry panel comprising 12 binding reagents that specifically recognize CD4, FOXP3⁺, CD49b, LAG-3, PD-1, Helios/USP22, CCR2, ST2, TIGIT, TOX/EOMES, CD127, and Areg to determine these regulatory and exhaust subsets.

In certain embodiments, the donor antigens are apoptotic donor leukocytes (ADLs), donor-specific transfusions, leukocytes expressing one or more donor MHC antigens mismatched with the recipient, leukocytes expressing one or more MHC class II antigens shared with the recipient, nanoparticles conjugated with donor peptides or encapsulating donor peptides, immature recipient-derived dendritic cells, and/or apoptotic recipient leukocytes conjugated with donor peptides. In certain embodiments the antigens, for example autoantigens like insulin or GADD65 or viral antigens for example viral capsid proteins, are incorporated in the apoptotic cells to induce tolerance in autoimmunity and gene therapy settings respectively. In certain embodiments, the donor antigens are formulated as donor-specific transfusion (DST) nanoparticles conjugated with donor peptides or encapsulating donor peptides, and/or apoptotic recipient leukocytes conjugated with donor peptides.

In certain embodiments, the transplant recipient patient has transplantation tolerance maintained. In certain embodiments, the transplant recipient patient had immune tolerance induced but failed. In certain embodiments, immune tolerance was not induced in the transplant recipient patient.

In certain embodiments, the target cells comprise Treg cells having markers CCR2^{hi}TIGIT^{hi}FOXP3⁺. Detection of only CCR2^{hi}TIGIT^{hi}FOXP3⁺ Treg cell in the patient's blood is sufficient to indicate the transplant tolerance because these Treg cells are 27-fold higher in the peripheral blood of tolerant animals in comparison to non-tolerant ones.

In certain embodiments, the present invention provides a method of identifying a transplant recipient patient having transplantation tolerance induced by peritransplant infusions (i.e., infusions around the time of transplant; with at least one infusion taking place days prior to the transplant) of apoptotic donor leukocytes under the cover of transient immunotherapy, comprising: (a) assaying a first blood sample from the patient to detect a baseline frequency of target cells, wherein the first blood sample is obtained pre-tolerization, pre-transplant, and pre-initiation of transient immunotherapy, (b) assaying a second blood sample from the patient to detect a post-procedure frequency of target cells, wherein the second sample is obtained post-tolerization, post-transplant, and post-initiation of transient immunotherapy; and (c) identifying the patient as having transplantation tolerance/immune acceptance induced by apoptotic donor leukocytes when the post-procedure frequency is at least 1.5-fold greater than the baseline frequency, wherein the target cells are wherein the target cells are T regulatory (Treg) cells having one or more combination of markers Helios^{hi}USP22^{hi}, CCR2^{hi}TIGIT^{hi} CCR2^{hi}TIGIT^{hi}Areg⁺, and/or CD4⁺CD25⁺CD127 FOXP3⁺; and/or wherein the target cells are T regulatory Type 1 (Tr1) cells having one or more combination of markers PD-1^{hi}CD69^{hi}Helios^{hi}, TIGIT^{hi} CCR2^{hi}ST2^{hi}GrnzB⁺FOXP3⁻, Areg^{hi} TIGIT^{hi} CCR2^{hi} GrnzB⁺ ST2^{hi} FOXP3⁻ and/or CD4⁺CD49b⁺LAG3⁺. GrnzB⁺TIGIT^{hi} CCR2^{hi}ST2^{hi} circulatory Tr1 cells are 10-fold higher in the peripheral blood of tolerant animals compared to non-tolerant ones. As such, GrnzB⁺ TIGIT^{hi}CCR2^{hi}ST2^{hi}FOXP3⁻ circulatory Tr1 cells are a reliable biomarker for transplant tolerance.

In certain embodiments, the Tr1 cells have indirect specificity for at least one mismatched donor MHC class I peptide (verified using recipient-specific MHC class-II tetramers loaded with said MHC class I peptides), have a transcriptomic signature indicative of antigen-specific signaling, and/or have a transcriptomic signature indicative of an activated state.

In certain embodiments, the target cells are wherein the target cells are T regulatory (Treg) cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127 FOXP3⁺; and/or wherein the target cells are T regulatory Type 1 (Tr1) cells having one or more combination of markers PD-1^{hi}CD69^{hi}Helios^{hi}, TIGIT^{hi} CCR2^{hi}ST2^{hi} GrnzB⁺FOXP3⁻, Areg^{hi} TIGIT^{hi} CCR2^{hi} GrnzB⁺ ST2^{hi} FOXP3⁻ and/or CD4⁺CD49b⁺LAG3⁺, have indirect specificity for at least one mismatched donor MHC class I peptide, have a transcriptomic signature indicative of antigen-specific signaling, and have a transcriptomic signature indicative of an activated state.

In certain embodiments, the target cells are wherein the target cells are exhaust T cells (Tex) cells having markers PD-1⁺ EOMES⁺ CD127^{hi} Helios⁺TOX⁺ TCF-1⁻, have indirect specificity for at least one mismatched donor MHC class I peptide, have a transcriptomic signature indicative of antigen-specific signaling, and have a transcriptomic signature indicative of an hypofunctional or exhaust state. In certain embodiments, the transplant recipient patient having transplantation tolerance induced by donor antigen administered under cover of transient immunotherapy has transplantation tolerance maintained.

As used herein, the term “under the cover of transient immunotherapy” means that the recipient transiently receives immunotherapy agents, such as immunosuppression drugs that target, among other cells, antigen presenting cells and their activation of donor-reactive T cells, any CD40 expressing cell, and T and B cells directly. As used herein “transient” means that the effects of the therapy lasts only for a short time, such as for a few days (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days), or for a few weeks (e.g., 1, 2, 3, 4, 5, 6, 7, 8, 9, 10, 11, or 12 weeks), or for a few months (e.g., 1, 2, 3, 4, 5, 6, 7, 8, 9, 10, 11, or 12 months). As used herein, “immunosuppression” means the partial (or modulation or modification) or complete suppression of the immune response, wherein the body's immune system is intentionally stopped from working, or is made less effective, than when the body is not receiving an immunosuppressive drug. In certain embodiments, the immunotherapy also includes the transient administration of anti-inflammatory therapies.

In certain embodiments, the present invention provides a method, comprising: (a) obtaining a first blood sample from a transplant recipient patient to detect a baseline frequency of target cells, wherein the first blood sample is obtained pre-tolerization, pre-transplant, and pre-initiation of transient immunotherapy, (b) obtaining a second blood sample from the patient to detect a post-procedure frequency of target cells, wherein the second sample is obtained post-tolerization, post-transplant, and post-initiation of transient immunotherapy, (c) assaying the first and second blood samples to detect levels of target cells before and after tolerization, (d) identifying the patient as having transplantation tolerance/immune acceptance induced by apoptotic donor leukocytes when the post-procedure frequency is at least 1.5-fold greater than the baseline frequency, wherein the target cells are wherein the target cells are T regulatory (Treg) cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻FOXP3⁺; and/or wherein the target cells are T regulatory Type 1 (Tr1) cells having one or more combination of markers PD-1^{hi}CD69^{hi}Helios^{hi}, TIGIT^{hi} CCR2^{hi}ST2^{hi} GrnzB⁺FOXP3⁻, Areg^{hi} TIGIT^{hi} CCR2^{hi} GrnzB⁺ ST2^{hi} FOXP3⁻ and/or CD4⁺CD49b⁺LAG3⁺; and/or wherein the target cells are exhaust CD4 T cells (Tex) having markers PD-1⁺ EOMES⁺ CD127^{hi} Helios⁺TOX⁺ TCF-1⁻.

In certain embodiments, the Tr1 and/or Treg cells have indirect specificity for at least one mismatched donor MHC class I peptide, have a transcriptomic signature indicative of antigen-specific signaling, and/or have a transcriptomic signature indicative of an activated state. In certain embodiments, the target cells are T regulatory Type 1 (Tr1) cells having one or more combination of markers PD-1^{hi}CD69^{hi}Helios^{hi}, TIGIT^{hi} CCR2^{hi}ST2^{hi}GrnzB⁺FOXP3⁻, Areg^{hi} TIGIT^{hi} CCR2^{hi} GrnzB⁺ ST2^{hi} FOXP3⁻ and/or CD4⁺CD49b⁺LAG3⁺, have indirect specificity for at least one mismatched donor MHC class I peptide, have a transcriptomic signature indicative of antigen-specific signaling, and have a transcriptomic signature indicative of an activated state. In certain embodiments, the target cells are T regulatory (Treg) cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻FOXP3⁺. In certain embodiments, the target cells are hypofunctional exhaust CD4 T cells (Tex) having marker PD-1⁺ EOMES⁺ CD127^{hi} Helios⁺TOX⁺ TCF-1⁻.

In certain embodiments, the present invention provides a method of treating a transplant recipient, the method comprising: (a) identifying the transplant recipient patient having transplantation tolerance/immune acceptance induced by apoptotic donor leukocytes (ADLs) infused under cover of transient immunotherapy using the method described above, and (b) treating the transplant recipient patient by ceasing to administer immunosuppressants.

In certain embodiments, the Tr1 cells exhibit indirect specificity for at least one mismatched donor MHC class I peptide.

In certain embodiments, the target cells are T regulatory Type 1 (Tr1) cells having markers PD-1^{hi}CD69^{hi}Helios^{hi}.

In certain embodiments, the target cells are T regulatory Type 1 (Tr1) cells having markers GrnzB⁺TIGIT^{hi} CCR2^{hi}ST2^{hi} FOXP3⁺.

In certain embodiments, the target cells are T regulatory (Treg) cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻FOXP3⁺. In certain embodiments, the target cells are T regulatory (Treg) cells having markers CCR2^{hi}TIGIT^{hi}FOXP3.

In certain embodiments, the target cells comprise a population of cells that are Tr1 cells having markers PD-1^{hi}CD69^{hi}Helios^{hi} and Treg cells having one or more combination of markers Helios^{hi}USP22^{hi} and/or CCR2^{hi}TIGIT^{hi} and CCR2^{hi}TIGIT^{hi}Areg.

In certain embodiments, the target cells comprise a population of cells that are hypofunctional exhaust CD4 T cells (Tex) having markers PD-1⁺ EOMES⁺ CD127^{hi} Helios⁺ TOX⁺ TCF-1⁻.

In certain embodiments, the target cells are spleen cells or lymph node cells.

In certain embodiments, the frequency of PD-1^{hi}CD69^{hi}Helios^{hi} and/or TIGIT^{hi} CCR2^{hi}ST2^{hi}GrnzB⁺FOXP3⁻ and/or Areg^{hi} TIGIT^{hi} CCR2^{hi} GrnzB⁺ ST2^{hi} FOXP3⁻Tr1 cells of all CD4 T-cells in step (a) is determined by multiparametric flow cytometry.

In certain embodiments, the frequency of PD-1^{hi}CD69^{hi}Helios^{hi}, TIGIT^{hi} CCR2^{hi}ST2^{hi} GrnzB⁺FOXP3⁻, Areg^{hi} TIGIT^{hi} CCR 2^{hi}GrnzB⁺ ST2^{hi} FOXP3⁻ and/or CD4⁺CD49b⁺LAG3⁺ Tr1 cells of all CD4⁺ T-cells in step (a) is determined by CyTOF mass cytometry.

In certain embodiments, the frequency of Helios^{hi}USP22^{hi}, CCR2^{hi}TIGIT^{hi}, CCR2^{hi}TIGIT^{hi}Areg⁺, and/or CD4⁺CD25⁺CD127⁻FOXP3⁺ Treg cells of all CD4⁺ T-cells in step (a) is determined by multiparametric flow cytometry. In certain embodiments, the flow cytometry is spectral flow cytometry.

In certain embodiments, the frequency of CCR2^{hi}TIGIT^{hi}FOXP3⁺ Treg cells of all CD4 T-cells in step (a) is determined by multiparametric flow cytometry. In certain embodiments, the flow cytometry is spectral flow cytometry.

In certain embodiments, the frequency of Helios^{hi}USP22^{hi}, CCR2^{hi}TIGIT^{hi}, CCR2^{hi}TIGIT^{hi}Areg⁺, and/or CD4⁺CD25⁺CD127⁻FOXP3⁺ Treg cells of all CD4⁺ T-cells in step (a) is determined by CyTOF mass cytometry.

In certain embodiments, the frequency of CCR2^{hi}TIGIT^{hi}FOXP3⁺ Treg cells of all CD4 T-cells in step (a) is determined by CyTOF mass cytometry.

In certain embodiments, the present invention provides kit comprising a panel of binding reagents, wherein the reagents individually specific for CD4, CD127, FOXP3 (i.c.), Areg⁺, CD49b, LAG-3, ST2, CCR2, PD-1, TOX/EOMES (i.c.), TIGIT, and Helios and/or USP22.

In certain embodiments, the binding reagents are antibodies.

In certain embodiments, the kit is a single-tube format.

In certain embodiments, the kit is compatible with BD/FACS and Cytek Aurora.

In certain embodiments, the kit further comprising preloaded gating templates for Treg, Tr1, Tex Tol modules.

In certain embodiments, the preloaded gating templates for the Treg module comprises % CD4⁺FoxP3⁺CD127low×USP22/Helios, CCR2 MFI.

In certain embodiments, the preloaded gating templates for the Tr1, module comprises % CD4⁺FOXP3⁻CD49b⁺LAG-3⁺×AREG expression.

In certain embodiments, the preloaded gating templates for the Tex Tol module % CD4⁺PD-1⁺CD127hi TIGIT⁺TOX⁺ (±EOMES) vs TIGIT-TOX-PD-1+/CD4 (activated T cell).

### DETAILED DESCRIPTION OF THE INVENTION

Negative vaccination with apoptotic donor leukocytes (ADLs) represents a promising, nonchimeric strategy for inducing donor antigen-specific tolerance in transplantation. Leukocytes treated ex vivo with the chemical cross-linker ethylcarbodiimide (ECDI) underwent rapid apoptosis after intravenous infusion. In murine allotransplant models, intravenous infusions of ECDI-treated apoptotic donor splenocytes on days-7 and +1 (relative to transplant on day 0) induced robust and alloantigen-specific tolerance to minor antigen-mismatched skin grafts, to fully major histocompatibility complex (MHC)-mismatched islet allografts, and, when combined with short-term rapamycin, to heart allografts. Most donor ECDI-treated splenocytes were quickly internalized by splenic marginal zone antigen presenting cells (APCs), whose maturation after uptake of apoptotic bodies was arrested, resulting in selective upregulated negative, but not positive, costimulatory molecules.

After encountering recipient APCs, T cells with indirect allospecificity rapidly increased in number, followed by profound clonal contraction; the remaining T cells were sequestered in the spleen, without trafficking to allografts or allograft-draining lymph nodes. Residual donor ECDI treated splenocytes that were not internalized by host phagocytes weakly activated T cells with direct allospecificity, rendering them resistant to subsequent stimulation (anergy). ECDI-treated splenocytes also activated and increased the number of regulatory T (Treg) and myeloid-derived suppressor cells (MDSCs). Thus, in murine allotransplant models, mechanisms of graft protection induced by alloantigen delivery via ECDI-treated splenocytes involved clonal anergy of antidonor CD4⁺ T cells with direct specificity, clonal depletion of antidonor CD4⁺ T cells with indirect specificity, and regulation by CD4⁺ Treg cells and MDSCs.

In murine models of autoimmunity and allergy, intravenous delivery of antigens cross-linked with ECDI to the surface of syngeneic leukocytes restored antigen-specific tolerance. Importantly, that strategy prevented both priming of naïve T cells and effectively controlled responses of existing memory/effector CD4⁺ and CD8⁺ T cells. A clinical trial involving multiple sclerosis patients affirmed the safety of intravenous delivery of encephalitogenic peptides after ECDI-coupling to autologous leukocytes, also yielding preliminary evidence of efficacy.

In previous studies on ECDI-treated donor splenocytes in murine allografts, we demonstrated stable tolerance to islet allografts in rhesus macaques (referred to as monkeys) given 2 ADL infusions under transient immunosuppression. We found that lasting tolerance in our model was associated with depletion of donor-specific T and B cell clones and, most prominently in recipients of 1 MHC class II (MHC-II) allele-matched ADL and allografts, potent and sustained regulation. Several immune cell subsets, including antigen-specific Tr1 cells, participated in immune regulation, suppressing posttransplant expansion of donor reactive T cells and their recruitment to allografts.

Transplantation tolerance induced by ADLs is associated with a sustained increase of regulatory immune cell subsets, including Tr1 cells and abundance of hypofunctional and/or exhaust T cells with distinct specificities and transcriptomic signatures, thereby identifying a biomarker for monitoring the induction, maintenance, and loss of regulatory tolerance induced by ADLs infused intravenously under the cover of transient immunosuppression. Greater than 1.5-fold increased frequency between baseline and post-procedure blood samples of CD49b⁺ LAG-3⁺ of circulating CD4⁺ T cells (Tr1 cells) that exhibit indirect specificity for at least 1 mismatched donor MHC class I peptide and transcriptomic signatures indicative of antigen specific signaling (e.g., SH2D2a) and mitochondrial respiration associated with an activated state (e.g., NDUFS4) is indicative of transplantation tolerance.

In certain embodiments, the present invention provides a method of identifying a transplant recipient patient having transplantation tolerance induced by peritransplant infusions of apoptotic donor leukocytes under the cover of transient immunotherapy, comprising: a method of identifying a transplant recipient patient having transplantation tolerance induced by peritransplant infusions of apoptotic donor leukocytes under the cover of transient immunotherapy, comprising: (a) assaying a first blood sample from the patient to detect a baseline frequency of target cells, wherein the first blood sample is obtained pre-tolerization, pre-transplant, and pre-initiation of transient immunotherapy, (b) assaying a second blood sample from the patient to detect a post-procedure frequency of target cells, wherein the second sample is obtained post-tolerization, post-transplant, and post-initiation of transient immunotherapy; and (c) identifying the patient as having transplantation tolerance/immune acceptance induced by apoptotic donor leukocytes when the post-procedure frequency is at least 1.5-fold greater than the baseline frequency, wherein the target cells are T regulatory (Treg) cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻FOXP3⁺ and/or wherein the target cells are T regulatory Type 1 (Tr1) cells having one or more combination of markers PD-1^{hi}CD69^{hi}Helios^{hi}, TIGIT^{hi} CCR2^{hi}ST2^{hi}GrnzB⁺FOXP3⁻, Areg^{hi} TIGIT^{hi} CCR2^{hi} GrnzB⁺ ST2^{hi} FOXP3⁺; and/or CD4⁺CD49b⁺LAG3⁺. In certain embodiments, the Tr1 cells have indirect specificity for at least one mismatched donor MHC class I peptide (verified using recipient-specific MHC class-II tetramers loaded with said MHC class I peptides), have a transcriptomic signature indicative of antigen-specific signaling, and/or have a transcriptomic signature indicative of an activated state.

In certain embodiments, the present invention provides a method, comprising: (a) obtaining a first blood sample from a patient to detect a baseline frequency of target cells, wherein the first blood sample is obtained pre-tolerization, pre-transplant, and pre-initiation of transient immunotherapy, (b) obtaining a second blood sample from the patient to detect a post-procedure frequency of target cells, wherein the second sample is obtained post-tolerization, post-transplant, and post-initiation of transient immunotherapy, (c) assaying the first and second blood samples to detect levels of target cells before and after tolerization, (d) identifying the patient as having transplantation tolerance/immune acceptance induced by apoptotic donor leukocytes when the post-procedure frequency is at least 1.5-fold greater than the baseline frequency, wherein the target cells are wherein the target cells are T regulatory (Treg) cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻FOXP3⁺, and/or wherein the target cells are T regulatory Type 1 (Tr1) cells having one or more combination of markers PD-1^{hi}CD69^{hi}Helios^{hi}, TIGIT^{hi} CCR2^{hi}ST2^{hi}GrnzB⁺FOXP3⁻, Areg^{hi} TIGIT^{hi} CCR2^{hi} GrnzB⁺ ST2^{hi} FOXP3⁻ and/or CD4⁺CD49b⁺LAG3⁺; and/or wherein the target cells are exhaust CD4 T cells (Tex) having markers PD-1⁺ EOMES⁺ CD127^{hi} Helios⁺TOX⁺ TCF-1⁻.

In certain embodiments, the Tr1 cells have indirect specificity for at least one mismatched donor MHC class I peptide, have a transcriptomic signature indicative of antigen-specific signaling, and/or have a transcriptomic signature indicative of an activated state.

In certain embodiments, the present invention provides a method of treating a transplant recipient patient, the method comprising: (a) identifying the transplant recipient patient as described herein and (b) treating the transplant recipient patient by ceasing to administer immunosuppressants.

It is important to determine if a transplant recipient patient is acquiring and maintaining immune tolerance to the transplant. In certain embodiments, the transplant that the patient received will be an allotransplant. As used herein, the term “allotransplant” is defined as a transplant of cells, tissues, or organs to a recipient from a genetically non-identical (i.e., distinct) donor of the same species. The transplant may be called an allograft, allogeneic transplant, or homograft. In certain embodiments, the allotransplant is a solid organ allotransplant, such as a kidney, pancreas, liver, intestine, heart, lung, or uterus transplant. In certain embodiments, the allotransplant is a tissue allotransplant, including but not limited to adipose tissue, amniotic tissue, chorionic tissue, connective tissue, dura, facial tissue, gastrointestinal tissue, glandular tissue, hepatic tissue, muscular tissue, neural tissue, ophthalmic tissue, pancreatic tissue, pericardia, skeletal tissue, skin tissue, urogenital tissue, and vascular tissue. In certain embodiments, the allotransplant is a cellular allotransplant, such as an islet, hepatocyte, myoblast, embryonic stem cell-derived differentiated cell transplant (e.g., islet or islet beta cell or hepatocyte transplant), or an induced pluripotent (following either transcription-factor-based programming or chemical reprogramming) stem cell-derived differentiated cell transplant (e.g., islet, islet beta cell or hepatocyte transplant), hematopoietic stem cell transplant, or bone marrow transplant.

As used herein, “immune acceptance,” “immune tolerance,” “immunological tolerance,” or “immunotolerance” is a state of unresponsiveness of the immune system to substances or tissue that have the capacity to elicit an immune response in given organism. The term “transplantation tolerance” is a form of immune tolerance. “Transplantation tolerance” is the long-term allograft survival in the absence of maintenance immunosuppressive therapy. Implicit to this definition is that tolerant recipients of organ transplants are unresponsive to donor antigens but maintain reactivity to other (third-party) antigens. Organ transplant recipients who have been successfully weaned from immunosuppression and have maintained stable graft function for 1 year or more are referred to as functionally or operationally tolerant.

### Immunotherapies

In certain circumstances, the transplant recipient patient will have received an immune therapy prior to, concurrently with, or subsequent to transplant, in order to induce transplantation tolerance, where the immune therapy is the administration of apoptotic donor leukocytes (ADLs).

In certain embodiments, the patient received immunotherapy prior to, concurrently with, or subsequent to a transplant. In certain embodiments, apoptotic donor leukocytes can be administered with, or in addition to, one or more immunomodulatory molecules such as antagonistic anti-CD40 mAb antibody, Fc-engineered anti-CD40L antibodies, a peptide interfering with CD40:CD40L co-stimulation, mTOR inhibitor (e.g., sirolimus, everolimus), and transient anti-inflammatory therapy including compstatin (e.g., the compstatin derivative APL-2), cytokine antagonists (e.g., anti-IL-6 receptor mAb (tozilizumab), anti-IL-6 antibody (sarilumab, olokizumab), soluble TNF receptor (etanercept), anti-TNF-alpha antibodies (e.g., infliximab (Remicade), adalimumab (Humira)), al-antitrypsin, nuclear factor-KB inhibitors (e.g., dehydroxymethylepoxyquinomycin (DHMEQ)), ATG (anti-thymocyte globulin) and other polyclonal T cell-depleting antibodies, alemtuzumab (Campath), anti-IL-2R Abs (basiliximab), B-cell targeting strategies (e.g., B cell depleting biologic, for example, a biologic targeting CD20, CD19, or CD22, and/or B cell modulating biologic, for example, a biologic targeting BLyS, BAFF, BAFF/APRIL, CD40, IgG4, ICOS, IL-21, B7RP1), mycophenolate mofetil, mycophenolic acid, down-regulators of down regulating sphingosine-1 phosphate receptors (e.g., FTY720), JAK inhibitors (e.g., tofacitinib), immunoglobulin (e.g., IVIg), CTLA4-Ig (Abatacept/Orencia), belatacept (LEA29Y, Nulojix), tacrolimus (Prograf), cyclosporine A, leflunomide, anti-CXCR3 antibody, anti-ICOS antibody, anti-OX40 antibody, anti-OX40L antibody, and anti-CD122 antibody, deoxyspergualin, soluble complement receptor 1, cobra venom factor, complement inhibitors (e.g., C1 inhibitor, compstatin), anti C5 antibody (eculizumab/Soliris), methylprednisolone, azathioprine.

In certain embodiments, the transient immunotherapy comprises at least one immunosuppressant. In certain embodiments, the immunosuppressant is an inhibitor of CD40:CD40L co-stimulation, an mTOR inhibitor, and concomitant anti-inflammatory therapy targeting proinflammatory cytokines. In certain embodiments, the inhibitor of CD40:CD40L co-stimulation is an antagonistic anti-CD40 antibody, an Fc-engineered (disabled, silent) or Fab′ anti-CD40L antibody, or a peptide interfering with CD40:CD40L co-stimulation. In certain embodiments, the inhibitor of CD40:CD40L co-stimulation is antagonistic anti-CD40 mAb 2C10R4. In certain embodiments, at least one immunosuppressant is sirolimus (Rapamycin). In certain embodiments, the transient immunotherapy comprises an anti-inflammatory agent. In certain embodiments, the anti-inflammatory agent is anti-IL-6R (tocilizumab) and/or sTNFR (etanercept).

In certain embodiments, to prevent activation of the immune system and induction of anti-donor immunity by the infusion of apoptotic donor leukocytes on days-7 and +1 relative to the transplant on day 0, the recipients were transiently immunosuppressed with drugs that target, among other cells, antigen presenting cells and their activation of donor-reactive T cells, other CD40-expressing cells, or T and B cells directly. Methods of preparing and administering apoptotic donor leukocytes is known in the art. Luo X, Pothoven K L, McCarthy D, DeGutes M, Martin A, Getts D R, Xia G, He J, Zhang X, Kaufman D B, Miller S D, ECDI-fixed allogeneic splenocytes induce donor-specific tolerance for long-term survival of islet transplants via two distinct mechanisms. Proc Natl Acad Sci USA. 2008 Sep. 23; 105 (38): 14527-32; Miller et al. U.S. Pat. No. 8,734,786. The first dose of each immunosuppressant was given on day −8 or −7 relative to the transplant on day 0. The antagonistic anti-CD40 mAb 2C10R4 was given IV at 50 mg kg⁻¹ on days −8, −1, 7, and 14. Rapamycin (Rapamune®) was given PO from day −7 through day 21 posttransplant; the target trough level was 5 to 12 ng mL-1. Concomitant anti-inflammatory therapy consisted of i) αIL-6R (tocilizumab, Actemra®) at 10 mg kg⁻¹ IV on days −7, 0, 7, 14, and 21, and ii) sTNFR (etanercept, Enbrel®) at 1 mg kg⁻¹ IV on days-7 and 0 and 0.5 mg kg⁻¹ SC on days 3, 7, 10, 14, and 21. Singh A, et al. Nature Communications. 2019 Aug. 2; 10 (1): 3495. In certain embodiments, the procedure followed is that described in US Patent Publication US 2022/0112280.

In certain embodiments, a first dose of immunosuppressant is administered to the patient seven to fourteen days before transplant (e.g., −1, −2, −3, −4, −5, −6, −7, −8, −9, −10, −11, −12, −13, −14 days). In certain embodiments, a second dose of immunosuppressant is administered to the transplant recipient patient a few days after transplant (e.g., day +1, +2, +3, +4, +5, +6, +7, +8, +9, +10, +11, +12, +13, +14, +15, +16, +17, +18, +19+, or +20). In certain embodiments, multiple doses of immunosuppressant are administered to the transplant recipient (e.g., 2, 3, 4, 5, 6, 7, 8, 9, or 10 doses) in the span of the treatment period of a few days to a few months.

The therapies can be administered through a chosen route of administration. The therapy may be administered intravenously, intraperitoneally or intramuscularly by infusion or injection.

### Target Cells

In certain embodiments of the present invention, a first (baseline) biological sample, such as a blood sample, is obtained from the patient prior to immune therapy and transplantation (“pre-tolerization”). In certain embodiments, on day −7 the patient receives an infusion of apoptotic donor cells, on day 0 the transplant recipient patient receives the transplant, and on day +1 the transplant recipient patient receives a second infusion of apoptotic donor cells. A second biological sample is obtained after transplantation (“post-transplant”), and a third sample is obtained after the second infusion of apoptotic donor cells. In certain embodiments, a fourth biological sample is obtained after the first infusion of cells and the transplant. From these samples, very specific target cells are isolated, namely, cells that are identified as wherein the target cells are T regulatory (Treg) cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻FOXP3⁺, and/or wherein the target cells are T regulatory Type 1 (Tr1) cells having markers USP22^{hi}Helios^{hi}CD69^{hi}. In certain embodiments, the Tr1 and/or Treg cells have indirect specificity for at least one mismatched donor MHC class I peptide, have a transcriptomic signature indicative of antigen-specific signaling, and/or have a transcriptomic signature indicative of an activated state. Tolerogenic Tr1 cells are a subset of CD4⁺ T cells that are thought to be an important mediator of tolerance/immune acceptance induced by the peritransplant infusions of apoptotic donor leukocytes.

Major histocompatibility complex (MHC) class II tetramer staining enables the characterization, quantification and sorting of defined, antigen-specific CD4⁺ T cells. MHC tetramers are an essential tool for characterizing antigen-specific CD4⁺ T cells. Protocols for the ex vivo tetramer staining of comparatively rare antigen-specific CD4⁺ T cells have provided a crucial tool for T-helper-cell analysis in basic and clinical immunology. (Uchtenhagen, H. et al. Efficient ex vivo analysis of CD4+ T-cell responses using combinatorial HLA class II tetramer staining. Nat. Commun. 7, 12614 (2016); Day, C. L. et al. Ex vivo analysis of human memory CD4 T cells specific for hepatitis C virus using MHC class II tetramers. J. Clin. Invest. 112, 831-842 (2003); Kwok, W. W. et al. Direct ex vivo analysis of allergen-specific CD4p T cells. J. Allergy Clin. Immunol. 125, 1407-1409.e1401 (2010); Moon, J. J. et al. Naive CD4+ T cell frequency varies for different epitopes and predicts repertoire diversity and response magnitude. Immunity 27, 203-213 (2007)). Accordingly, MHC class II tetramer staining has become an invaluable approach in immunology, enabling direct interrogation of the naturally developing T-cell repertoire, assessment of changes in T-cell responses caused by perturbations such as vaccination and disease, and providing a means of confirming the translational relevance of observations in model systems.

It is known in the art to identify CD4⁺ T-cells that have markers Helios^{hi}USP22^{hi} and/or CCR2^{hi}TIGIT^{hi} using flow cytometry and gating. These CD4⁺ T-cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻FOXP3⁺ are referred to as Treg cells. In certain embodiments, the frequency of Treg cells in the samples is determined by multiparametric flow cytometry. In certain embodiments, the flow cytometry is spectral flow cytometry. A subset of Treg cells with indirect specificity are identified using MHC class II tetramers loaded with mismatched donor MHC class I peptides. In certain embodiments, this powerful tetramer technology tracks these rare and donor peptide-specific cell subsets. In certain embodiments, the frequency of Treg cells is determined by CyTOF mass cytometry.

It is known in the art to identify CD4⁺ T-cells that have markers PD-1^{hi}CD69^{hi}Helios^{hi}, TIGIT^{hi} CCR2^{hi}ST2^{hi}GrnzB⁺FOXP3⁻, Areg^{hi} TIGIT^{hi} CCR2^{hi} GrnzB⁺ ST2^{hi} FOXP3⁻ and/or CD4⁺CD49b⁺LAG3⁺ using flow cytometry and gating. These CD4⁺ T-cells are referred to as Tr1 cells. In certain embodiments, the frequency of Tr1 cells in the samples is determined by multiparametric flow cytometry. A subset of Tr1 cells with indirect specificity are identified using MHC class II tetramers loaded with mismatched donor MHC class I peptides. In certain embodiments, this powerful tetramer technology tracks these rare and donor peptide-specific cell subsets. In certain embodiments, the frequency of Tr1 cells is determined by CyTOF mass cytometry.

Next, the specific target cells, i.e., Treg cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻FOXP3 and/or Tr1 CD4⁺ T-cells that having one or more combination of markers PD-1^{hi}CD69^{hi}Helios^{hi}, TIGIT^{hi} CCR2^{hi}ST2^{hi}GrnzB⁺FOXP3⁻, Areg^{hi} TIGIT^{hi} CCR2^{hi} GrnzB⁺ ST2^{hi} FOXP3⁻ and/or CD4⁺CD49b⁺LAG3⁺ were analyzed to determine if they have indirect specificity for at least one mismatched donor MHC class I peptide. This determination of the presence of at least one mismatched donor MHC class I peptide is generated using multiparametric flow cytometry. In certain embodiments, the mismatched donor MHC class I peptide is APVALRNLRGYYNQS (SEQ ID NO:1), a 14-mer peptide in the variable region of the MHC class I molecule (28-114 aa). A t-BLAST analysis was performed of the Mamu DRB sequence with the human genome at the NCBI website to determine the human homolog. HLA DRB1*13 (Acc. No. CDP32905.1) was 92% identical, with 96% positives and 0% gaps to the Mamu DRB03a with an e value of 6e-178 and HLA DRB1*14 (Acc. No. ABN54683.1) was 93% identical, with 94% positives and 0% gaps to the Mamu DRB04 with an e value of 2e-175.

In certain embodiments, the target cells are spleen cells or lymph node cells, graft tissues, bronchoalveolar lavage (BAL), or urine samples.

Peptides from Mamu MHC class I and class II sequence with high binding affinity for HLA DRB1*13 or HLA DRB1*14 were identified (Table 1) using the Immune Epitope Database Analysis resource.

TABLE 1
MHC class I Peptides that bind to MHC Class II molecule.

| | Source | | | |
|---|---|---|---|---|
| Tetramer | Antigen | Sequence | Position | SEQ ID NO. |
| HLA-DRB1*14:01 | Mamu-A4 | APVALRNLRGYYNQS | 98 | 1 |
| HLA-DRB1*14:01 | Mamu-A8 | SLRYFYTAVSRPGRG | 28 | 2 |
| HLA-DRB1*14:01 | Mamu-A8 | TRIYKAATQNYREGL | 88 | 3 |
| HLA-DRB1*14:01 | Mamu-A1 | SMKYFYTSMSRPGRG | 28 | 4 |
| HLA-DRB1*14:01 | Mamu-A1 | WEPFSQSTIPMVGII | 298 | 5 |
| HLA-DRB1*14:01 | Mamu-A2/49 | SMRYFYTSMSRPGRW | 28 | 6 |
| HLA-DRB1*03:01 | Mamu-A4 | TQFVRFDSDAASQRM | 55 | 7 |
| HLA-DRB1*03:01 | Mamu-A8 | TQFVRFDSDAESPRE | 55 | 8 |
| HLA-DRB1*03:01 | Mamu-A2 | APVNLRNLRGYYNQS | 98 | 9 |
| HLA-DRB1*14:01 | Mamu-A2 | APVNLRNLRGYYNQS | 98 | 10 |
| HLA-DRB1*03:01 | Mamu-DR3a | YVRFDSDVGEHRAVS | 66 | 11 |
| HLA-DRB1*14:01 | Mamu-DR4 | GAGLFIYFRNQKGPS | 243 | 12 |
| HLA-DRB1*14:01 | Mamu-DRla | GAGLFIYFRNQKGHT | 243 | 13 |

The specific target cells were also analyzed to determine their transcriptomic signature indicative of antigen-specific signaling. As used herein the “transcriptomic signature” of a cell is the expression level of RNAs in a cell population. Briefly, RNA from the sorted target cells is analyzed by using quantitative real-time PCR using a set of primers and probes selected and defined by previous unbiased RNAseq analyses of cells from transplant recipients with documented and stable tolerance. In certain embodiments of the present invention, quantitative real-time PCR was done on RNA obtained from flow-sorted Tr1 cells. In certain embodiments, the transcriptomic signature indicative of antigen-specific signaling is SH2 Domain Containing 2A (SH2D2a).

TABLE 2
Differentially Expressed Transcripts in Trl cells.

| | | |
|---|---|---|
| | EDGE test: cohort | EDGE test: cohort |
| | B vs cohort C, | B vs cohort C, |
| | tagwise dispersions - | tagwise dispersions - |
| Feature ID | Fold change | P value |

Immune Activation

| | | |
|---|---|---|
| ABI2 | 191.78 | 1.89E−03 |
| ANAPC11 | 98.37 | 3.75E−03 |
| BATF | 76.83 | 8.13E−03 |
| CCR5 | 20.49 | 4.87E−03 |
| CD300E | 92.87 | 4.38E−03 |
| CYB5R3 | 17.44 | 2.21E−03 |
| DSC3 | 63.55 | 2.04E−03 |
| HMGB1 | 20.04 | 7.53E−03 |
| IFNLR1 | 43.08 | 8.41E−03 |
| ISG15 | 61.67 | 8.45E−03 |
| MAPK7 | 31.08 | 5.49E−03 |
| MBL2 | 1024.92 | 2.22E−03 |
| NANOG | 60.09 | 7.55E−03 |
| NCK1 | 30.04 | 5.07E−03 |
| POLR3K | 72.95 | 9.27E−03 |
| PROS1 | 187.13 | 4.81E−03 |
| SH2D2A | 21.11 | 3.54E−03 |
| SLC27A2 | 93.21 | 5.46E−03 |
| TOM1 | 31.54 | 1.41E−03 |
| TRIM68 | 33.12 | 4.01E−03 |
| TUBB2B | 53.27 | 2.74E−03 |

Signal Transduction

| | | |
|---|---|---|
| ABI2 | 191.78 | 1.89E−03 |
| ACKR1 | 1136.81 | 3.96E−03 |
| ARHGEF38 | 1008.09 | 2.19E−03 |
| CCR5 | 20.49 | 4.87E−03 |
| CNKSR2 | 356.54 | 7.04E−03 |
| CTTN | 87.42 | 2.73E−03 |
| DISP2 | 183.45 | 2.61E−03 |
| DLG3 | 450.87 | 2.29E−04 |
| GAB1 | 72.27 | 5.04E−03 |
| GFRA1 | 107.2 | 7.10E−03 |
| HIST1H4L | 488.58 | 5.07E−03 |
| ITGB3 | 259.36 | 5.00E−03 |
| LRP5 | 183.98 | 5.35E−03 |
| MAPK7 | 31.08 | 5.49E−03 |
| MCF2L | 391.04 | 4.75E−04 |
| MIS12 | 90.09 | 6.24E−03 |
| NCBP2 | 17.43 | 6.87E−03 |
| NCK1 | 30.04 | 5.07E−03 |
| P2RY2 | 235.71 | 4.15E−03 |
| PMEPA1 | 95.68 | 5.75E−03 |
| PRKAG1 | 17.32 | 7.96E−03 |
| RGS16 | 39.93 | 3.99E−03 |
| RGS18 | 215.98 | 3.55E−03 |
| RNF2 | 218.7 | 3.32E−03 |
| SH2D2A | 21.11 | 3.54E−03 |
| SKA2 | 25.53 | 5.64E−03 |
| TUBB2B | 53.27 | 2.74E−03 |

Metabolism

| | | |
|---|---|---|
| ACBD4 | 56.83 | 9.42E−03 |
| ACOT7 | 72.59 | 9.50E−03 |
| ADI1 | 88.84 | 3.70E−03 |
| ADO | 35.39 | 1.81E−03 |
| COQ2 | 88.53 | 6.22E−03 |
| CYB5R3 | 17.44 | 2.21E−03 |
| CYP2C8 | 356.35 | 9.30E−03 |
| GDPD5 | 99.21 | 4.19E−03 |
| GK | 82.51 | 9.38E−03 |
| ISCA1 | 47.9 | 3.13E−03 |
| LIPT1 | 72.99 | 9.35E−03 |
| MCEE | 23.58 | 4.40E−03 |
| MMADHC | 35.8 | 6.00E−03 |
| MTRR | 13.47 | 8.76E−03 |
| NDUFB1 | 29.03 | 8.62E−03 |
| NDUFS4 | 27.47 | 8.19E−03 |
| PFKP | 16.23 | 4.22E−03 |
| PHKA1 | 42.34 | 2.30E−03 |
| PRKAG1 | 17.32 | 7.96E−03 |
| RPL7 | 1100.39 | 1.29E−04 |
| RPSA | 28.9 | 5.69E−03 |
| SGMS2 | 498.32 | 7.37E−03 |
| SLC27A2 | 93.21 | 5.46E−03 |
| SULT4A1 | 512.47 | 5.82E−03 |
| UGCG | 25.35 | 5.83E−03 |
| UGT2A1 | 356.35 | 9.30E−03 |
| ZDHHC21 | 90.5 | 6.61E−03 |

Gene Expression

| | | |
|---|---|---|
| E2F8 | 173.1 | 4.67E−03 |
| HIST1H4L | 488.58 | 5.07E−03 |
| MOBP | 694.57 | 8.32E−03 |
| MYBL2 | 20.93 | 7.45E−03 |
| NCBP2 | 17.43 | 6.87E−03 |
| PLAGL1 | 20.18 | 8.92E−03 |
| POLR3K | 72.95 | 9.27E−03 |
| PRKAG1 | 17.32 | 7.96E−03 |
| RNF2 | 218.7 | 3.32E−03 |
| SNAPC5 | 82.01 | 7.02E−03 |
| TTF1 | 15.69 | 6.13E−03 |
| ZNF181 | 20 | 8.94E−03 |
| ZNF253 | 71.19 | 9.18E−03 |
| ZNF398 | 19.22 | 8.55E−03 |
| ZNF426 | 69.53 | 3.97E−03 |
| ZNF441 | 35.1 | 2.29E−03 |
| ZNF684 | 70.74 | 9.26E−03 |
| ZNF688 | 134.9 | 3.00E−03 |
| ZNF75D | 26.53 | 7.43E−03 |

In certain embodiments, the transcriptomic signature is indicative of an activated state. In certain embodiments, the transcriptomic signature indicative of an activated state is mitochondrial respiration-associated transcript NADH: Ubiquinone Oxidoreductase Subunit S4 (NDUFS4).

### Transplants

In certain embodiments, the transplant is an allotransplant. In certain embodiments, the allotransplant is a solid organ allotransplant. In certain embodiments, the allotransplant is a solid organ allotransplant, such as a kidney, pancreas, liver, intestine, heart, lung, or uterus transplant. In certain embodiments, the solid organ allotransplant is a kidney transplant. In certain embodiments, the allotransplant is a tissue allotransplant, including but not limited to adipose tissue, amniotic tissue, chorionic tissue, connective tissue, dura, facial tissue, gastrointestinal tissue, glandular tissue, hepatic tissue, muscular tissue, neural tissue, ophthalmic tissue, pancreatic tissue, pericardia, skeletal tissue, skin tissue, urogenital tissue, and vascular tissue. In certain embodiments, the allotransplant is a cellular allotransplant, such as an islet, hepatocyte, myoblast, embryonic stem cell-derived differentiated cell transplant (e.g., islet or islet beta cell or hepatocyte transplant), or an induced pluripotent stem cell-derived differentiated cell transplant (e.g., islet or islet beta cell transplant), hematopoietic stem cell transplant, or bone marrow transplant.

In certain embodiments, the transplant is a living donor transplant. In certain embodiments, the allotransplant is a cellular transplant.

### Assay Methods

In certain embodiments, the present invention involves the steps of (a) assaying a first blood sample from a patient to detect a baseline frequency of target cells, wherein the first blood sample is obtained pre-tolerization, pre-transplant, and pre-initiation of transient immunotherapy, (b) assaying a second blood sample from the patient to detect a post-procedure frequency of target cells, wherein the second sample is obtained post-tolerization, post-transplant, and post-initiation of transient immunotherapy; and (c) identifying the patient as having transplantation tolerance/immune acceptance induced by apoptotic donor leukocytes when the post-procedure frequency is at least 1.5-fold greater than the baseline frequency, wherein the target cells are T regulatory Type 1 (Tr1) cells, defined as CD49b⁺, LAG-3⁺, CD4⁺ cells. In certain embodiments, the Tr1 cells have indirect specificity for at least one mismatched donor MHC class I peptide, have a transcriptomic signature indicative of antigen-specific signaling, and/or have a transcriptomic signature indicative of an activated state.

In certain embodiments, the frequency of target cells in the first (baseline) sample is compared to the frequency of target cells in the second (post-procedure) sample and subsequent samples. In certain embodiments, the determination of at least a 1.5-fold increase in the frequency indicates tolerance/immune acceptance induced by the peritransplant infusion of apoptotic donor leukocytes. In certain embodiments, the determination of at least a 1.5-fold increase in the frequency indicates tolerance/immune acceptance induced by the peritransplant infusion of apoptotic donor leukocytes. In certain embodiments, the frequency between the first and second sample is at least 2× increase, at least a 3× increase, at least a 4× increase, at least a 5× increase, at least a 10× increase, at least a 20× increase, at least a 30× increase, at least a 50× increase, at least a 60× increase, at least a 70×, at least a 80× increase, at least a 90× increase, at least a 100×, or higher-fold increase.

In certain embodiments, the frequency of the target cells is determined by multiparametric flow cytometry. In certain embodiments, the flow cytometry is spectral flow cytometry. In certain embodiments, the frequency of the target cells is determined by CyTOF mass cytometry.

In certain embodiments, the transplant recipient patient has received two peritransplant, intravenous infusions of apoptotic donor leukocytes.

In certain embodiments, peripheral blood mononuclear cells from the transplant recipient patient is stained with a defined cocktail of fluorescence-conjugated antibodies and markers (anti-CD4, anti-CD49b, anti-LAG3, MHC class-II tetramer loaded with mismatched donor MHC class I peptides) to facilitate sorting of labeled cells using microfluidic technology, and the labeled cells are characterized using subsequent quantitation of the tolerance-associated transcripts using quantitative real time PCR.

The percentage of such defined CD4⁺ CD49b⁺LAG3⁺ T cells (Tr1 cells) with indirect specificity for mismatched donor peptides and expressing transcripts indicating antigen-specific TCR signaling (SH2D2a) and indicating a metabolically active state (NDUFS4)) is exceedingly low at baseline. A more than 1.5-fold increase in the percentage of that circulating T cell subset cannot be explained other than the presence of an antigen-specific tolerant state.

The invention will now be illustrated by the following non-limiting Examples.

### Example 1

Allospecific Splenic Tr1 Cells Drive Effector T Cell Exhaustion Through Upregulated Areg-EGFR Signaling to Promote Transplant Tolerance

### Abstract

Inducing stable tolerance to transplants remains a challenge in immunology. Previously, we induced tolerance to allogeneic islets in nonhuman primates by preemptive alloantigen delivery to antigen-presenting cells in situ. Here, mass cytometry phenotyping with incorporated donor-derived MHC-I peptide-loaded MHC-II tetramers revealed accumulation of allospecific CD4⁺ T cell clusters in the spleen of tolerant recipients. Areg Tr1 regulatory and terminally exhausted EGFR^{hi} T (Tex) cells represented the predominant allospecific subsets. Trajectory analysis showed that antigen-experienced effector memory T cells differentiated into suppressive Areg⁺Tr1 and EGFR⁺TOX⁺Nur77⁺TCF-1⁻ Tex subsets. Cell-cell communication mapping showed that exhausted and effector memory T cells engaged with allospecific Tr1 cells via the Areg-EGFR axis. Gene silencing studies confirmed that Tr1 cells utilize Areg-EGFR signaling to drive the metabolic suppression and epigenetic reprogramming of CD4⁺ T cells through a Nur77-dependent pathway. These findings point to the splenic Areg⁺Tr1 cell-EGFR⁺Teff cell axis as a critical immunoregulatory pathway in peripheral transplant tolerance.

### Introduction

Pancreatic islet transplants can restore near-normal blood glucose control in individuals with type 1 diabetes. However, the overall benefits of this therapy are often compromised by the toxic side effects of the immunosuppressive drugs required to prevent graft rejection. To enable widespread adoption of islet replacement therapies, it is essential to develop strategies that eliminate the need for long-term immunosuppression.

To address this challenge, we induced immune tolerance to allogeneic islet transplants in nonhuman primates (NHPs) by preemptively delivering alloantigen in the form of apoptotic donor leukocyte (ADL) infusions under induction immunosuppression. This strategy resulted in sustained, drug-free survival of islet allografts. The mechanisms of tolerance we documented included deletion of alloreactive T cells, expansion of IL-10-producing type 1 regulatory (Tr1) cells, and recruitment of Tr1 cells to the allograft. According to prior studies, efferocytosis of cells in early apoptosis elicits robust anti-inflammatory and immunoregulatory effects on APCs. This process facilitates the deletion or reprogramming of antigen-experienced T cells into Foxp3⁺ regulatory T cells (Tregs) and IL-10-secreting Tr1 cells that promote peripheral tolerance to antigens derived from the engulfed apoptotic material. Gaining deeper insights into the microenvironments and mechanisms that drive the differentiation of Tr1 cells—and their ability to modulate effector T cell (Teff) responses—could significantly advance the clinical translation of islet replacement therapies that do not require maintenance immunosuppression.

In murine models of allergy, autoimmunity, graft-versus-host disease (GVHD), and transplantation, Tr1 cells mediate tolerance by suppressing Teff cell responses through IL-10, TGF-β, cytolytic activity, and CD39/CD73-mediated metabolic disruption. Whether Tr1 cells contribute to islet allograft tolerance in NHPs by inducing exhaustion or metabolic restraint, akin to Tregs in mice, remains unclear. T cell exhaustion, characterized by diminished cytokine output and sustained inhibitory receptor expression, supports graft survival in murine studies and in clinical kidney transplant recipients; it also plays important roles in infection, cancer, and autoimmunity. Metabolic restraint—defined by impaired mitochondrial function, glycolysis, amino acid metabolism—reduces nutrient uptake and T cell fitness, thereby limiting effector activity, proliferation, and immune activation.

The mode and route of antigen delivery critically shape the immunological context in which the antigen is encountered. By modulating the functional programming of antigen-presenting cells (APCs), these factors influence the nature of APC-T cell interactions. Inflammatory microenvironments favor APC-T cell crosstalk that drives effector T cell responses and pathogen clearance, whereas anti-inflammatory settings promote peripheral tolerance through the induction of regulatory T cell subsets. Intravenously administered apoptotic leukocytes predominantly localize to the spleen, where they are rapidly internalized and processed by splenic APCs.

Efferocytosis of early apoptotic cells by APCs, particularly by splenic marginal zone macrophages and conventional type 1 dendritic cells (cDC1s), plays a pivotal role in the induction of immune tolerance. In both macrophages and cDC1s, efferocytosis activates liver X receptor (LXR)-dependent transcriptional programs that drive their differentiation toward a tolerogenic phenotype. These tolerogenic macrophages and cDC1s are characterized by reduced co-stimulatory molecule expression and enhanced regulatory cytokine production. Tolerogenic DCs are well-established inducers of Tr1 cells and play a pivotal role in the establishment of antigen-specific immune tolerance. For these reasons, profiling immune cell signatures within the spleen may uncover previously unrecognized mechanisms of tolerance induction relevant to our transplant model.

To uncover mechanisms driving sustained tolerance, we interrogated the regulatory and exhaustion signatures, developmental trajectories, phenotypic heterogeneity, intercellular crosstalk, and functional programs of allospecific CD4⁺ T cells in NHPs that rejected or tolerized transplanted islets after immunosuppression withdrawal. To monitor allospecific CD4 T cells, we employed high-dimensional mass cytometry with incorporated MHC class II tetramers loaded with mismatched donor MHC class I peptides. Tracking these cells across spleen and peripheral blood lymphocytes (PBLs), we uncovered tolerance-associated regulatory, exhaustion, and metabolic restraint programs. We also analyzed the role of amphiregulin (Areg), a ligand of epidermal growth factor receptor (EGFR) and a potent enhancer of Treg stability and inhibitor of inflammation. We demonstrated that allospecific Tr1 cells and hypofunctional exhausted CD4⁺ T cells (Tex) coexpressing Areg and EGFR were significantly enriched in the spleen of tolerant NHPs. The spleen of tolerant NHPs emerged as a key site for effector fate decisions, where effector-naïve cells appeared to shift toward exhaustion and regulatory programs. Our further mechanistic studies revealed that Tr1 cells used Areg-EGFR signaling to drive the metabolic suppression and epigenetic reprogramming of CD4⁺ T cells through a Nur77-dependent pathway. Together, our findings identified the splenic Areg⁺Tr1-EGFR⁺Teff axis as a key regulatory circuit in sustaining peripheral transplant tolerance.

### Results

### Tolerance is Linked to Distinct CD4-T Cell Clusters in the Spleen

We identified 26 phenotypically distinct CD4⁺ T cell clusters with unique marker expression and compartmental distribution. We annotated these clusters as naïve, memory, regulatory, or exhausted subsets, according to canonical markers and curated phenotypes (Tables 3-7).

TABLE 3
Study Cohort ID and tissues analyzed

| | Animal ID | Cohort | Experimental group | Tissues | FCS file |
|---|---|---|---|---|---|
| 1 | 15CP6 | B | Control_1DR Match | PBL | A2 |
| | | | | Spleen | A2 |
| 2 | 15CP3 | B | Control_1DR Match | NA | |
| | | | | Spleen | A4 |
| 3 | 14HP34 | B | Control_1DR Match | PBL | A5 |
| | | | | Spleen | A5 |
| 4 | 14HP31 | B | Control_1DR Match | PBL | A6 |
| | | | | Spleen | A6 |
| 5 | 13EP5 | C | ADL_1DR match | PBL | C1 |
| | | | | Spleen | C1 |
| 6 | 15CP4 | C | ADL_1DR match | PBL | C2 |
| | | | | Spleen | C2 |
| 7 | 15CP1 | C | ADL_1DR match | PBL | C3 |
| | | | | Spleen | C3 |
| 8 | 15FP01 | D | ADL_1DR mismatch | PBL | D1 |
| | | | | Spleen | D1 |
| 9 | 15FP03 | D | ADL_1DR mismatch | PBL | D3 |
| | | | | Spleen | D3 |
| 10 | 13EP11 | N | Naive/Healthy | PBL | N1 |
| | | | | Spleen | N1 |
| 11 | 15CP14 | N | Naive/Healthy | PBL | N2 |
| | | | | Spleen | N2 |
| 12 | 15CP12 | N | Naive/Healthy | PBL | N3 |
| | | | | Spleen | N3 |

TABLE 4
CyTOF Panel

| S.N. | Mass Tag | Marker | Clone |
|---|---|---|---|
| | 89Y | CD45 | HI30 |
| 1 | 106Cd | CD45RA | 5H9 |
| 2 | 110Cd | CD3 | SP34-2 |
| 3 | 111Cd | CD95 | DX2 |
| 4 | 112Cd | CCR2 | 48607 |
| 5 | 113Cd | CD45RO | UCHL |
| 6 | 114Cd | CD4 | L200 |
| 7 | 116Cd | CD45 | D058-1283 |
| 8 | 141Pr | CD62L | Dreg56 |
| 9 | 142Nd | OX40 | ACT35 |
| 10 | 143Nd | ICOS | C398.4A |
| 11 | 144Nd | CD69 | FN50 |
| 12 | 145Nd | Amphiregulin | AREG559 |
| 13 | 146Nd | Neuropilin | 12C2 |
| 14 | 147Sm | GrzB | GB-11 |
| 15 | 148Nd | CD49b | AK-7 |
| 16 | 149Sm | CD25 | M-A251 |
| 17 | 150Nd | CD127 | HIL-2R-M21 |
| 18 | 151Eu | USP22 | EPR4352(2) |
| 19 | 152Sm | RNF20 | EPR13563(B) |
| 20 | 153Eu | Tim-3 | F38-2E2 |
| 21 | 154Sm | GITR | 621 |
| 22 | 155Gd | PD-1 | EH12-2H7-BL |
| 23 | 156Gd | Helios | 22F6 |
| 24 | 158Gd | CCR4 | L291H4 |
| 25 | 159Tb | SATB1 | O96C6 |
| 26 | 160Gd_anti-FITC | FITC-Tetramer:Non- | |
| | | specific peptides | |
| 27 | 161Dy | Tbet | eBio4B10 |
| 28 | 162Dy | FoxP3 | 206D |
| 29 | 163Dy | | |
| 30 | 164Dy | TCF-1 | 7F11A10 |
| 31 | 165Ho_anti-PE | PE-Tetramer:MHCl | |
| | | peptides | |
| 32 | 166Er | TOX | 6E6D03 |
| 33 | 167Er | EGFR | WD1928 |
| 34 | 168Er | Eomes | H11 |
| 35 | 169Tm | CD8 | RPT8 |
| 36 | 170Er | ST2 | hIL33Rcap |
| 37 | 171Yb | CCR7 | 150503 |
| 38 | 172Yb | CD103 | 2G5.1 |
| 39 | 173Yb | GARP | 7B11 |
| 40 | 174Yb | HLA-DR | L243 |
| 41 | 175Lu | Nur77 | REA704 |
| 42 | 176Yb | LAG-3 | Polyclonal |
| 43 | 191/193Ir | Cells Size | |
| 44 | 194/198Cis | Viability | |
| 45 | 209Bi | TIGIT | MBSA43 |

TABLE 5
Tetramers used in CyTOF panel for comprehensive characterization of CD4⁺ T cells

| Loaded on | | | | | | |
|---|---|---|---|---|---|---|
| Tetramer-PE | Animal ID | Cohorts | Tetramer | Source Antigen | Sequence | Position |
| Tetramer 7-PE | 15CP6 | B | HLA-DRB1*14:01 | Mamu-A8 | SLRYFYTAVSRPGRG | 28 |
| Tetramer 12-PE | 15CP3 | B | HLA-DRB1*03:01 | Mamu-A4 | TQFVRFDSDAASQRM | 55 |
| Tetramer 14-PE | 14HP34 | B | HLA-DRB1*03:01 | Mamu-A2 | APVNLRNLRGYYNQS | 98 |
| Tetramer 11-PE | 14HP31 | B | HLA-DRB1*14:01 | Mamu-A2/49 | SMRYFYTSMSRPGRW | 28 |
| Tetramer 13-PE | 13EP5 | C | HLA-DRB1*03:01 | Mamu-A8 | TOFVRFDSDAESPRE | 55 |
| Tetramer 14-PE | 15CP4 | C | HLA-DRB1*14:01 | Mamu-A2 | APVNLRNLRGYYNOS | 98 |
| Tetramer 8-PE | 15CP1 | C | HLA-DRB1*14:01 | Mamu-A8 | TRIYKAATQNYREGL | 88 |
| Tetramer 9-PE | 15FP01 | D | HLA-DRB1*14:01 | Mamu-A1 | SMKYFYTSMSRPGRG | 28 |
| Tetramer 10-PE | 15FP02 | D | HLA-DRB1*14:01 | Mamu-A1 | WEPFSQSTIPMVGII | 298 |
| Tetramer 11-PE | 15FP03 | D | HLA-DRB1*14:01 | Mamu-A2/49 | SMRYFYTSMSRPGRW | 28 |

Sequences are SEQ ID NO: 13-23 in order of appearance in Table 5

TABLE 6
Resolutions that accurately determine the cluster
number for Total CD4⁺ T cells and subsets.

| | Reso- | |
|---|---|---|
| CD4 T cells subsets | lutions | No. of clusters |
| Total CD4 T cells | 0.4 | 26, (c0-c25)* |
| | | *clusters |
| | | representing <0.0002 |
| | | (c26-c30) are excluded |
| Tet (MHCl)⁺ CD4 T cells | 0.6 | 16, (c0-c15), lowest |
| (Allospecific CD4 T cells) | | is 0.0037 |
| Tet(MHCl)⁺ PD-1^{hi} CD4 T cells | 1 | 10, (c0-c9), lowest |
| (Allospecific Tex cells) | | is 0.017 |
| Tet(MHCl)⁺ CD127^{low} FoxP3⁺CD4 | 0.6 | 10, (c0-c9), lowest |
| T cells (Allospecific Treg cells) | | is 0.0063 |
| Tet(MHCl)⁺ CD49b⁺LAG-3⁺ CD4 | 0.6 | 9, (c0-c8), lowest |
| T cells (Allospecific Tr1 cells) | | is 0.0273 |

TABLE 7
Top differentially expressed (DE) genes in each cluster of total CD4⁺ T cells

| Clusters | CD4⁺ T cell subsets | DE Genes | | | | | | |
|---|---|---|---|---|---|---|---|---|
| 0 | CD95^{hi} effector memory | CD95 | CCR4 | ICOS | | | | |
| 1 | Terminally exhaust | TOX | CD62L | RNF20 | Areg | | | |
| 2 | CD69^{hi} Effector | CD69 | PD-1 | TIGIT | ICOS | | | |
| 3 | CD127^{hi} memory | CD127 | CCR7 | SATB1 | CD103 | | | |
| 4 | CCR4^{hi} memory | CCR4 | LAG-3 | CCR7 | | | | |
| 5 | GrzB^{hi} effector | GranzB | | | | | | |
| 6 | Temra | CD45RA | CCR7- | HLA-DR | CD95 | CCR2 | CD62L- | |
| 7 | Tcm | LAG-3 | GITR | ST2 | | | | |
| 8 | Tbet^{hi} effector | Tbet | Helios | | | | | |
| 9 | Helios^{hi} activated Tregs | FoxP3 | Helios | USP22 | EGFR | EOMES | Nur77 | CD127 |
| 10 | TIM-3^{hi} Tex | TIM-3 | | | | | | |
| 11 | PD-t^{hi} Tex | PD-1 | TIGIT | ICOS | ST2 | TCF-1łow | | |
| 12 | Tmemo | LAG-3 | CD25 | GARP | CD127 | CD103 | CD62L | CCR2 |
| 13 | Tact. memo | CD49b | TCF-1 | | | | | |
| 14 | TCF-1^{hi} Tregs | FoxP3 | TCF-1 | | | | | |
| 15 | Tmemo | CD103 | CD95 | CCR4 | CD45RO | | | |
| 16 | Act. Areg⁺ memory | CD69 | ICOS | OX40 | Areg | NLRP | | |
| 17 | Areg⁺ activated Tregs | FoxP3 | EOMES | EGFR | LAG-3 | CD45RO | Areg | NLRP |
| 18 | TOX^{hi} Tex | TOX | EOMES | | | | | |
| 19 | NLRP^{hi} Tregs | FoxP3 | NLRP | TOX | | | | |
| 20 | TIGIT^{hi} ST2⁺ Treg | TIGIT | LAG-3 | CCR2 | CD62L | CD45RA | ST2 | FoxP3 |
| 21 | CCR2^{hi} Tregs | CD45RO | CCR2 | TOX+ | TCF-1 | | | |
| 22 | TIM-3^{hi} Tex | TIM-3 | PD-1 | TIGIT | EGFR | GITR | | |
| 23 | CCR4^{hi} activated | OX40 | CCR4 | CD49b | | | | |
| 24 | Activated memory | GARP | HLA-DR | CD103 | | | | |
| 25 | Activated memory | CD25 | CD49b | CD127 | | | | |

Among these clusters, seven (c1, c9, c14, c17, c19-c21) displayed regulatory T cell features and four (c10, c11, c18, c22) exhaustion-like phenotypes. The differences were notable between those Cohorts. The remaining clusters encompassed naïve, activated, and effector memory T cell (Tem) subsets. Notably, cluster c1 with Tr1 features was enriched 3.4-fold in PBLs and 6.5-fold in the spleen of tolerant-Cohort C vs. nontolerant-Cohort B, with substantial increases also observed relative to Cohorts D and N. Clusters c9 and c20-characterized by Helios, USP22, TIGIT, and ST2 expression-were substantially increased in Cohort C (vs. B, D and N). Specifically, in tolerant-Cohort C (vs. nontolerant-Cohort B) NHPs, cluster c9 was enriched 76.3-fold in the spleen; cluster c20, 41-fold in PBLs and 3.2-fold in the spleen. Exhausted clusters c10, c11, c18, and c22-marked by PD-1, TIM-3, TIGIT, and TOX-were more prevalent in tolerant-Cohort C: c22 was enriched 7.5-fold in PBLs and 3.2-fold in the spleen.

Conversely, Tem clusters c2-c7, c24, and c25 were enriched in the PBLs and spleen of nontolerant-Cohort B: c6 (Temra) by 4.6-fold in the spleen, indicating a rejection-associated phenotype. Our radar chart displayed elevation of EOMES and LAG-3 expression in cluster c1; of EOMES and Helios in cluster c9; and of TIM-3 and TOX in cluster c22. In nontolerant-Cohort B, the Temra cluster c6 displayed higher CD45RA and CD95 expression, signifying an effector phenotype. In nontolerant-Cohort C, the abundance of regulatory and exhausted T cell subsets and markers correlation in the spleen suggested a role of that compartment in maintaining tolerance.

### Allospecific CD4⁺ T Cell Clusters Accumulate in the Spleen of Tolerant NHPs

In our earlier work, we observed a low number of allospecific T cells in the PBLs of tolerant NHPs. That observation led us to question whether the tolerance protocol deleted these cells or simply relocated them to secondary lymphoid organs. Given that intravenously administered ADLs primarily distribute to the spleen compartment, we examined it for the presence and heterogeneity of allospecific CD4⁺ T cells. To do so, we used MHC class II tetramers loaded with mismatched donor-derived MHC-I peptides (II-Tet^{pMHC-I+}). In tolerant-Cohort C NHPs, the proportion of allospecific cells (II-Tet^{pMHC-I+}) among CD4⁺ T cells was higher in the spleen vs. PBLs, a pattern not observed in nontolerant-Cohorts B and D.

Among 16 phenotypically distinct subsets (Table 8), 8 clusters (c2, c4-c7, c11, c13, c15) exhibited regulatory features.

TABLE 8
Top differentially expressed (DE) genes in each cluster of total allospecific
(II-Tet^{pMHC-I+}) CD4⁺ T cells.

| | Phenotype of | | | | | | | | | |
|---|---|---|---|---|---|---|---|---|---|---|
| Cluster | allospecific CD4⁺ T cells | DE Genes | | | | | | | | |
| 0 | Tem or Tcm | CCR7 | CCR4 | CD127 | CD62L^{low} | | | | | |
| 1 | Act. Terminal TEM | CD95 | CD45RA | HLA-DR | CCR2 | CCR7 | CD62L- | | | |
| 2 | OX40^{hi} Tr1 cells | LAG-3 | OX40 | NRLP | CD62L | ST2 | CD49b | | | |
| 3 | Act. Teff exhaust | PD-1 | TIGIT | CD69 | ICOS | CD95 | | | | |
| 4 | Act. Tex | TOX | RNF20 | EOMES | Helios | Areg | Nur77 | CD62L | LAG-3 | TCF-1- |
| 5 | Act. Treg1 | USP22 | CD127 | Helios | Eomes | EGFR | Nur77 | FoxP3 | NLRP | |
| 6 | Act. Treg2 | TCF-1 | FoxP3 | Tbet | HLA-DR | GITR | CCR2 | CD45RO | CD95 | CD103 |
| 7 | Act. Treg3 | TCF-1 | FoxP3 | Tbet | | | | | | |
| 8 | TIM-3^{hi} Tex | Tim-3 | Nur77 | USP22 | | | | | | |
| 9 | TIGIT^{hi} Tex | TIGIT | CCR2 | ST2 | CD62L | RNF20 | Helios | LAG-3 | TCF-1 | |
| 10 | OX40^{hi} act. Tem | OX40 | ICOS | CD69 | SATB1 | Areg | CD62L | NRLP | | |
| 11 | Areg⁺ NRLP⁺ Treg | GmzB | NRLP | EGFR | Nur77 | Areg | Tbet | SATB1 | CD45RO | FoxP3 |
| 12 | HLA-DR^{hi} act. Tcm | CD45RO | CCR2 | HLA-DR | CD45RA | | | | | |
| 13 | CD127^{hi} act. Tr1 like | CD25 | CD49b | CD127 | GITR | LAG-3 | | | | |
| 14 | TOX^{hi}Tex | TOX | Eomes | EGFR | ST2 | | | | | |
| 15 | OX4^{hi} CCR4^{hi} act. Treg | OX40 | CCR4 | CD62L | Tbet | Helios | FoxP3 | | | |

The allospecific CD4⁺ T cell cluster c4—exhibited Tr1 cell features and coexpressed EOMES and Helios, was enriched in the spleen (11.9-fold) and PBLs (4.1-fold) in tolerant (vs. nontolerant) NHPs. Cluster c5 (Helios⁺USP22⁺), reflecting a proliferative, suppressive Treg phenotype, was enriched 27.8-fold in the spleen of tolerant (vs. nontolerant) NHPs. Tr1-cluster c15 (OX40^{hi}CCR4^{hi}Tbet⁺) was enriched about 7-fold in both the spleen and PBLs of tolerant (vs. nontolerant) NHPs.

We found that four allospecific CD4⁺ T cell clusters (c3, c8, c9, c14) exhibited canonical Tex signatures, including elevation of TIGIT and TOX. Cluster c3 (TIGIT^{hi}PD-1^{hi}) was comparably represented across Cohorts, but cluster c9 (TIGIT⁺LAG-3⁺TCF-1^{low}) was markedly enriched in tolerant NHPs: about 33-fold in the PBLs and about 3.7-fold in the spleen. The Tex-cluster c14 (TOX^{hi}EOMES^{hi}) was enriched 6-fold in the spleen of tolerant NHPs. In contrast, memory-like clusters (c1, c10, c12) were reduced in tolerant NHPs, while cluster c0 showed no enrichment in tolerant (vs. nontolerant) NHPs.

The accumulation of regulatory and exhausted allospecific CD4⁺ T cells in the spleen of tolerant NHPs led us to examine whether donor-specific effector function was constrained to this compartment. We assessed the polyfunctionality of splenic CD4⁺ T cells after stimulating them ex vivo with donor antigens. CD4⁺ T cells from the spleen of tolerant NHPs showed impaired polyfunctionality, with severely reduced IFN-γ, TNF-α, and IL-17 coexpression (Table 9). Collectively, these findings delineate a tolerance-associated signature in the spleen, 10 characterized by enrichment of regulatory and exhausted cells among allospecific CD4⁺ T cells.

TABLE 9
Flow cytometry panels: CD4⁺ T cell cytokine polyfunctionality
assay, Trl cells enumeration, CD4⁺ T cells exhaustion,
proliferation and histone acetylation.

| | | |
|---|---|---|
| Markers | Clone | Flourochrome |

Flow cytometry: T cell cytokine polyfunctionality

| | | | |
|---|---|---|---|
| 1 | IL-17 | eBio64DEC17 | FITC |
| 2 | IL-2 | MQ1-17H12 | PE |
| 3 | CD4 | Fox | PerCP |
| 4 | TNF-α | 1MAb11 | PECy7 |
| 5 | Perforin | PF-344 | APC/AF647 |
| 6 | IFN-γ | B27 | AF700 |
| 7 | Ghost Dye ™ UV 450 | | UV450 |
| 8 | CD3 | SP34-2 | BV510 |

Flow Panet: Tr1 cell enumeration

| | | | |
|---|---|---|---|
| 1 | CD3 | SP34-2 | FITC |
| 2 | LAG-3 | Polyclonal | PerCP |
| 3 | CD4 | L200 | AF700 |
| 4 | Ghost Dye ™ UV 450 | | UV450 |
| 5 | CD49b | AK-7 | BV510 |

Flow Panet: T cell exhaustion

| | | | |
|---|---|---|---|
| 1 | Nur77 | E6 | FITC |
| 2 | LAG-3 | Polyclonal | PerCP |
| 3 | TOX | | APC |
| 4 | TIGIT | 2629A | AF700 |
| 5 | Areg | Areg559 | PECy7 |
| | EGFR | H11 | PE |

Flow Panel: CFSE Flow-MLR

| | | | |
|---|---|---|---|
| 1 | CFSE | | FITC |
| 2 | CD4 | L200 | AF700 |
| 3 | Ghost Dye ™ UV 450 | | UV450 |
| 4 | CD3 | SP34-2 | BV510 |

Flow Panel: Histone acetylation

| | | | |
|---|---|---|---|
| 1 | H3k9ac | C5B11 | FITC |
| 2 | H3k27ac | D5E4 | AF647 |
| 3 | CD4 | L200 | AF700 |
| 4 | Ghost Dye ™ UV 450 | | UV450 |
| 5 | CD3 | SP34-2 | BV510 |

### Allospecific Tr1 Cells Engage Tex and Tem Cells Via the Areg-EGFR Axis in the Spleen of Tolerant NHPs

The increased frequency of Areg⁺ Tr1 and EGFR⁺ Teff cells in tolerant NHP spleens suggested regulatory-effector cell crosstalk. Our cell-cell interaction analysis using the CellChat algorithm revealed a dominant Areg-EGFR axis, linking Tr1 and Tex/Tem clusters, with dense, bidirectional signaling networks confined to tolerant NHPs. Tr1 cell clusters c2 and c4 exhibited the strongest outgoing signals, targeting Tex clusters c3, c8, and c9 and Teff-like clusters c0 and c1. This pattern—absent in nontolerant Cohorts B and D—aligned with elevated Areg⁺, EGFR, and immunoregulatory HELIOS, Nur77, and EOMES transcription factor expression profiles in the spleen of tolerant NHPs. Our heatmap analysis confirmed higher global communication probability, predominantly driven by Tr1 clusters.

### Allospecific Tr1 and Tex Cell Developmental Fates are Linked to Temra Cells in Tolerant Spleens

The accumulation of distinct Tr1 and Tex subsets in tolerant spleens, together with directional Areg-EGFR signaling, suggested a shared developmental origin. We applied Slingshot-based pseudotime analysis to allospecific CD4⁺ T cells from the spleen, using Temra cells (CD95⁺CCR7⁻CD45RA⁺; c1) as the origin, and identified nine trajectories resolving into four fates: Tex-like (c3, c8, c9, c14; lineages 1-3, 5), Tr1-like (c13, c15; lineages 4, 8), Treg-like (c7, c11; lineages 6, 9), and memory/effector-like (c12; lineage 7) phenotypes.

Lineages 4, matured into GrnzB^{hi}Areg⁺ (c13) Tr1-like cells; lineages 8 into OX40^{hi}CCR4^{hi}Areg⁺ (c15) Tr1-like cells. The higher pseudotime in Cohort C, indicated progressive regulatory commitment. Cohort C displayed increased Tr1-like pseudotime and terminal state occupancy (>65%), supporting a link between tolerance and regulatory T cell maturation. Lineage 6 matured into Tbet TCF-1⁺ (c7) and activated Treg-like cell fates that were more abundant in tolerant (vs. nontolerant) NHPs.

Lineages 1, 2, and 5 matured into distinct Tex-like states, characterized by TIGIT TIM-3⁺Nur77⁺ (c8), PD-1^{hi}TIGIT^{hi}LAG-3⁺CCR2^{hi} (c9), and EOMES⁺TIM-3⁺TOX⁺ (c14) phenotypes. Exhaustion trajectories were defined by progressive upregulation of TOX, TIGIT, and TIM-3 with loss of TCF-1. Pseudotime-based exhaustion scores were significantly higher in tolerant (vs. nontolerant) NHPs; 60 to 80% of Tex cells occupied terminal states. Cohort D showed intermediate differentiation, consistent with partial exhaustion. In summary, our pseudotime analysis revealed the transition of antigen-experienced CD4⁺ Temra cells into regulatory and exhausted states during tolerance.

### Areg⁺ Tr1 Cells are the Predominant Allospecific Regulatory Subset in the Spleen of Tolerant NHPs

Having shown the high maturity levels of allospecific (II-Tet^{pMHC-I+}) LAG-3⁺CD49b⁺ Tr1 cells in the spleen of tolerant NHPs, we next examined their phenotypic heterogeneity and fate trajectories. Allospecific Tr1 cells were significantly enriched in the spleen (vs. PBLs): a 2-fold increase in tolerant-Cohort C (vs. nontolerant-Cohort B). Unsupervised clustering revealed nine phenotypically distinct Tr1 subsets, including those expressing coinhibitory receptors (c0, c1, c3, c4, c8) and chemokine receptors (c0-c5, c8), aligning with signatures previously reported in autoimmune and inflammatory diseases. Additionally, we found clusters lacking canonical markers, suggesting functional diversity (Table 10).

TABLE 10
Top differentially expressed (DE) genes within each cluster of total allospecific
(II-Tet^{pMHC-I+}) LAG-3⁺ CD49b⁺ Tr1 cells.

| Cluster | Tr1 cell phenotype | DE Genes | | | | | | |
|---|---|---|---|---|---|---|---|---|
| 0 | PD-1^{hi}LAG-3⁺Tr1 | OX40 | ICOS | CD69 | Neuropilin | CD103 | GITR | PD-1 |
| 1 | CCR7^{hi}TOX⁺Helios⁺Tr1 | CCR7 | CCR4 | TOX | LAG-3 | CD49b | GmzB | |
| 2 | CD95^{hi}HLA-DR^{hi}eff.Tr1 | CD95 | HLA-DR | CCR2 | CD45RO | | | |
| 3 | PD-1^{hi}Heliosh^{hi} act.Tr1 | PD-1 | Helios | USP22 | CD69 | TOX | | |
| 4 | CCR2^{hi}TIGIT^{hi}ST^{hi}Tr1 | TIGIT | CCR2 | ST2 | RNF20 | CD62L | CD45RO | |
| 5 | TCF^{hi}Tbet⁺ act. Tr1 | TCF-1 | Tbet | GITR | HLA-DR | CCR2 | | |
| 6 | ICOS^{hi}CD69^{hi} Areg act. Tr1 | OX40 | ICOS | CD69 | SATB1 | Areg | Neuropilin | EGFR |
| 7 | EOMES^{hi} GmzB^{hi} Areg⁺ eff. Tr1 | Eomes | GmzB | Nur77 | SATB1 | Areg | Neuropilin | CD45RO |
| 8 | TIM-3^{hi}USP22⁺Nur77⁺ Tr1 | Nur77 | TIM-3 | USP22 | CCR4 | | | |

In tolerant NHPs, the allospecific Tr1 cell clusters c3, c4, c5, and c7 were enriched in the spleen (vs. PBLs), whereas cluster c4 was more abundant in PBLs. Cluster c3 was predominantly localized to the spleen in tolerant NHPs (76% of the c3): Areg, CD95, and CD69 expression along, with CCR7 coexpression, indicated migratory potential. Cluster c4 coexpressing Areg and EGFR was markedly expanded in Cohort C: 6-fold in the spleen, 21-fold in PBLs (vs. Cohort B); we also noted TIGIT, GrnzB, ST2, and CCR2 expression in Tr1 cells indicating activated states. Additional clusters (c5, c7) also displayed Areg⁺EGFR⁺ profiles with elevated Tbet, EOMES, and GrnzB. Trajectory inference defined four differentiation paths originating from c5 or c7 and converging into c1, c3, c4, or c7. Terminal pseudotime accumulation in c3 and c4 was most prominent in Cohort C, characterized by Areg⁺, Helios, Tbet, CD69, and HLA-DR expression, suggesting advanced Tr1 maturation. These findings identify the spleen as a key site for Areg⁺ Tr1 cell expansion and functional programming during transplant tolerance.

EGFR⁺ Terminally Exhausted Allospecific CD4⁺ T Cells Accumulate in the Spleen of Tolerant NHPs

Having demonstrated the accumulation of phenotypically mature allospecific Areg⁺ Tr1 cells in the spleen of tolerant NHPs, we next examined whether tolerance was similarly associated with the splenic accumulation of allospecific exhausted CD4⁺ T cells (Table 11). Given the spleen's role in peripheral tolerance and the link between chronic antigen exposure and T cell exhaustion, we analyzed II-Tet^{pMHC-I+} allospecific PD-1⁺CD4⁺Tex cells in the spleen and PBLs of NHPs. Total allospecific Tex cell frequencies were similar across Cohorts (˜10%). However, in tolerant NHPs Tex cell enrichment was 3-fold in the spleen (vs. PBLs), significantly higher than in nontolerant.

TABLE 11
Top differentially expressed (DE) genes within each cluster of total allospecific
(II-Tet^{pMHC-I+}) CD4⁺ Tex cells

| Cluster | Tex phenotype | DE Genes | | | | | | |
|---|---|---|---|---|---|---|---|---|
| 0 | CCR4^{hi}TOX⁺ Tem | CCR4 | CD69 | TOX | CCR7 | CD62L | TCF-1- | |
| 1 | CD127^{hi} Tcm | LAG-3 | CD127 | CD103 | CCR7 | CD62L | GITR | GARP |
| 2 | CD95^{hi} CCR2^{hi} Tem | CCR2 | CD95 | CD45RA | CD45RO | TCF-1 | HLA-DR | CCR7- |
| 3 | Helios^{hi} Tcm | Helios | HLA-DR | CCR7 | CD62L | | | |
| 4 | TIGIT^{hi} Terminally | TIGIT | PD-1 | CD69 | ICOS | TOX | TCF-1 | CD45RA |
| 5 | PD-1^{hi} Teff | CD95 | PD-1 | CD69 | ICOS | TCF-1 | TOX- | CCR7- |
| 6 | EOMES^{hi} Terminally | OX40 | CD62L | CCR4 | Nur77 | LAG-3 | Eomes | |
| 7 | TIM-3^{hi} act. Tcm | Tim-3 | Nur77 | TIGIT | CD69 | CD62L- | CD45RA | |
| 8 | Tbet^{hi} TCF-1⁺ Tcm | Tbet | TIGIT | ICOS | CD69 | CCR4 | TCF-1 | |
| 9 | OX40^{hi} ICOS^{hi} Tem | OX40 | ICOS | CD69 | CD62L | CCR7- | TOX- | TCF-1- |

Among ten phenotypically distinct clusters, most—except c2 and c5—were markedly expanded in the spleen of tolerant vs. nontolerant NHPs. Clusters c1 (11.4-fold), c3 (3.6-fold), c4 (15.5-fold), and c8 (3.7-fold) were substantially enriched in the spleen of Cohort C. Notably, cluster c1 (PD-1^{hi}EOMES⁺TOX⁺TCF-1⁻) and c4 (EGFR⁺TIGIT^{hi}PD-1⁺TCF-1⁻) represented terminally exhausted PD-1^{hi}TCF-1⁻ Tem-like cells. Conversely, clusters c2 and c5, enriched in nontolerant NHPs, retained TCF-1 and downregulated TOX, indicating a partially exhausted yet functionally preserved state. The coexpression analysis revealed positive correlations between exhaustion markers, transcription factors, and effector/memory phenotypes. Our pseudotime trajectory analyses further delineated four developmental paths originating from memory-like subsets (c2, c8, c9) and maturing into terminally exhausted Tex clusters (c1, c4). Tolerant NHPs showed higher pseudotime scores, with >65% of allospecific Tex cells occupying terminal exhaustion states, characterized by progressive TIGIT, TOX, and EGFR acquisition. In contrast, trajectories c8→c2 and c9→c5 showed higher pseudotime in Cohort B, retaining TCF-1 and low TOX, indicating preserved effector function despite exhaustion markers. Together, these data suggest that splenic Tex cells during tolerance mirror conserved exhaustion trajectories observed in cancer and chronic viral infection, supporting their role in immune restraint and in durable transplant tolerance.

### The Areg-EGFR Axis Mediates the Tr1-Driven Suppression and Metabolic Regulation of CD4⁺ T Cells

Prior studies have linked Areg to Treg cell stability as well as to Treg-mediated antiinflammatory functions, T cell differentiation, exhaustion, and tumor immune evasion. In tolerant NHPs, we observed a significant increase in Areg⁺Tr1 cells alongside EGFR-expressing Tex/eff cells, with strong cell-cell interactions between Tr1, Tex, and Teff cells in the spleen. Building on these findings, we sought to gain mechanistic insight into Areg's role in Tr1 cell mediated immune regulation. Specifically, we tested whether Areg promotes Tr1 differentiation and induces T cell exhaustion. Recombinant Areg treatment led to a >2-fold increase in the coexpression of Areg and EGFR in the CD4⁺ T cells, accompanied by upregulation of exhaustion-associated transcription factors Nur77 and TOX. In parallel, such treatment led to a 3- to 6-fold expansion of Tr1 cells, indicating a role for Areg in promoting Tr1 cell differentiation. Furthermore, we found that Areg exposure suppressed histone acetylation by ˜60% and substantially downregulated metabolic gene programs controlling glycolysis and mitochondrial respiration in CD4⁺ T cells.

To assess the functional contribution of Tr1 cell-derived Areg, we sorted Tr1 cells from ex vivo ADL-stimulated cultures, rested them overnight, and treated them for 72 hours with or without Areg-specific siRNA. We passively transferred these cells into one-way mixed lymphocyte reactions, transfecting responder T (respT) cells with and without Nur77 siRNA. Tr1 cells significantly suppressed CD4⁺ T cell proliferation, but Areg silencing in Tr1 cells or Nur77 knockdown in respT cells restored proliferation by 40% to 60%, indicating that Tr1-derived Areg contributes to over 50% of their suppressive function via a Nur77-dependent mechanism. Notably, the presence of Areg Tr1 cells markedly suppressed the oxygen consumption rate and downregulated key metabolic genes in responder T cells. In contrast, Areg silencing in Tr1 cells or Nur77 knockdown in respT cells abrogated that effect, resulting in a significant increase in the oxygen consumption rate and a 2-fold increase in metabolic gene expression. Tr1 cells suppressed histone acetylation in CD4 respT cells, as evidenced by reduced H3K9ac and H3K27ac protein expression. Areg silencing in Tr1 cells led to a >38% increase in acetylation; likewise, Nur77 knockdown in CD4⁺ respT cells also elevated acetylation by >30%, indicating that Areg-Nur77 signaling cooperatively enforces epigenetic restraint. Together, these findings establish the Areg⁺Tr1-EGFR⁺CD4⁺ Teff cell axis as a key mechanism of Tr1-mediated immune suppression and metabolic regulation, highlighting its contribution to transplant tolerance.

### Discussion

Tolerance to transplanted islets would facilitate the broad adoption of cell replacement therapies for patients with diabetes. In a prior NHP study, we showed that tolerance to transplanted islets was associated with the sustained expansion of circulating Tr1 cells. In our current study, we identified allospecific Areg⁺Tr1 cells in the spleen of NHPs as regulators of Teff cell exhaustion through Areg-EGFR signaling, thereby delineating a previously unrecognized mechanism by which Tr1 cells contribute to the maintenance of transplant tolerance.

Using high-dimensional mass cytometry and donor-specific MHC class II tetramers, we profiled allospecific CD4⁺ T cells from the spleen and PBLs posttransplant. We identified phenotypically distinct Tr1 and Tex cell populations with tolerance-associated enrichment of Areg⁺Tr1 and EGFR⁺ Tex cell subsets in the spleen. Our trajectory analysis—and functional perturbation of Areg in Tr1 cells and of Nur77 in responder T cells—revealed that Areg-EGFR signaling drives Tr1 differentiation and Teff exhaustion via Nur77-dependent metabolic and epigenetic reprogramming, thereby defining a regulatory axis sustaining transplant tolerance. Notably, tolerance to transplanted islets was sustained long-term in these NHPs, suggesting the spleen's critical role as a niche maintaining active immune regulation.

Allospecific Tr1 cells, defined as CD49b⁺LAG-3⁺/CD4⁺ T cells binding to MHC class II tetramers that present the mismatched donor-derived MHC class I peptides, were significantly enriched in the spleen, but not PBLs, of tolerant (vs. nontolerant) NHPs. Among nine transcriptionally distinct allospecific Tr1 clusters, five were significantly enriched in either PBLs or spleen of tolerant recipients. Notably, the CCR2^{hi} cluster c4 was the only one enriched in PBLs, while the CCR 7^{hi} cluster c3 was one of four clusters enriched in the spleen of tolerant (vs. nontolerant) NHPs. The higher abundance of splenic Areg^{hi} Tr1 cell clusters in tolerant NHPs suggests that tolerance is associated with selective expansion of tissue-resident, regulatory Tr1 populations. In addition to high expression of CCR7, these clusters coexpressed CD69, CD103, and GrnzB, suggesting a localized, tissue-resident splenic allospecific Tr1 cell subset with regulatory function that may help maintain transplant tolerance. The Areg^{hi}CD69^{hi}CD103⁺ phenotype of Tr1 cells suggests a spleen-tethered population that can locally modulate Teff cell fates. The elevated EOMES, USP22, Helios, and ST2 expression in splenic allospecific Tr1 cells suggests a tolerogenic program associated with their differentiation, stabilization, regulation enhancement, and Areg expression. Consistent with our findings, recent transcriptomic studies in human and murine Tr1 cells have identified GrnzB, TIGIT, TIM-3, and ICOS enrichment as part of their regulatory signature. Collectively, these findings identify the spleen as a conducive environment for Tr1 cell differentiation, expansion, and function in tolerant NHPs.

Considering the evolving role of T cell exhaustion in transplant tolerance, we explored the abundance and allospecificity of Tex cell phenotypes in the spleen of tolerant (vs. nontolerant) NHPs. The ten phenotypically distinct clusters of allospecific CD4⁺ Tex cells that we found displayed diverse expression profiles of inhibitory receptors, transcription factors, and the immunoregulatory molecules Areg and EGFR, with significantly higher enrichment of EGFR⁺ subsets in the spleen of tolerant (vs. nontolerant) NHPs. The selective enrichment of TOX^{hi}PD-1^{hi}TCF-1^{low} phenotypes in Tex clusters was associated with a profound reduction in polyfunctional donor-reactive CD4⁺ T cells in the spleen of tolerant NHPs. This constellation of markers defines terminal exhaustion, a state maintained by a TOX-driven exhaustion program. The observed phenotype mirrors exhaustion signatures described in human patients with chronic infection and cancer, suggesting that this conserved Tex differentiation trajectory is also operative in transplant tolerance.

Our trajectory analysis revealed that allospecific Tex cells in tolerant NHPs arise from memory-like precursors, consistent with prior studies showing that chronic antigen exposure drives exhaustion via NFAT, TOX, and NR4A transcriptional programs. Several exhaustion-related phenotypes observed in our study align with those previously reported in murine transplant models. In tolerant NHPs, exhaustion may be reinforced by persistent donor antigen, by anergy, and by an IL-10/Areg-rich immunosuppressive milieu.

Our interactome analyses suggest that the Areg-EGFR axis is implicated in Tr1 cell differentiation and Teff cell exhaustion. Areg-EGFR signaling is known to regulate T cell immunity and FoxP3⁺ Treg stability. Areg⁺, produced by Tregs, activates EGFR signaling in neighboring Tregs and DCs, reinforcing immune suppression and limiting Teff activation. Areg-EGFR signaling stabilizes FoxP3 in Tregs and enhances immune regulation, of potential relevance in Tr1 maintenance and Teff restraint. In our current study, Areg-EGFR signaling emerged as a defining feature of allospecific regulatory and exhausted CD4⁺ T cell states in tolerant NHPs.

Functionally, in vitro Areg stimulation of CD4 T cells upregulated both Areg and EGFR expression and enhanced Tr1 differentiation, implicating Areg-EGFR signaling as a direct regulator of Tr1 cell fate. Our cell-cell communication analysis revealed robust directional Areg-EGFR signaling from Tr1 clusters to EGFR Tex/Teff cells. These interactions, most pronounced in the spleen of tolerant NHPs, delineate a regulatory circuit in which Tr1 cells promote Teff exhaustion.

Our trajectory analysis revealed that Areg⁺EGFR⁺Tr1 cells (cluster c7), enriched 3.5-fold in the spleen of tolerant NHPs, originated from antigen-experienced Tem cells and progressed to mature Areg^{hi}Tr1 states. These findings complement earlier studies identifying effector memory CD4⁺ T cells as Tr1 precursors. Recent evidence supports the capacity of follicular helper T cells to give rise to Tr1 cells under pMHCII-NPs stimulation. Given the limited resolution of Tfh-defining markers in our dataset, it is plausible that Tfh-like cells were encompassed within the Temra pool. Our results support a model in which Tr1 cells arise from diverse antigen-experienced subsets, shaped by tolerogenic cues and cytokines such as IL-10 and IL-27, with EGFR signaling potentially reinforcing regulatory fate and stability through cooperation with transcriptional programs involving c-Maf, AHR, and Blimp-1.

Tex clusters c1 and c4, (marked by elevated EGFR, TIGIT, PD-1, TOX, and diminished TCF-1), were enriched >12-fold in the spleen of tolerant NHPs, suggesting sustained engagement of exhaustion programs. These populations coexpressed EOMES and Nur77, implicating EGFR signaling in shaping Tex differentiation during tolerance. In support of that implication, we found that Areg stimulation induced EGFR and Nur77 expression in CD4⁺ T cells and enhanced TOX upregulation, whereas Areg silencing reversed these effects. Collectively, these data suggest that Areg-EGFR signaling orchestrates the NFAT-TOX-Nur77 axis, establishing a transcriptional circuit that sustains allospecific CD4⁺ T cell exhaustion during transplant tolerance.

Our functional studies revealed that Areg stimulation enhanced Tr1 differentiation while inducing TOX and Nur77 and suppressing metabolic gene expression in CD4⁺ T cells. Silencing Areg in Tr1 cells or Nur77 in responder T cells reversed these effects and restored Teff proliferation, metabolism, and histone acetylation, (an epigenetic marker of activation), thus highlighting the role of Areg⁺Tr1 cells in metabolic and transcriptional reprogramming of Teff cells. These findings align with reports that Areg blockade enhances CD8 T cell expansion, reduces Treg frequencies, and increases inflammatory gene expression in myeloid cells. Furthermore, AREG deletion in CD4⁺ T cells or Tregs impairs tumor progression, supporting its broader immunosuppressive role. Areg has also been implicated in activating TGF-β and reducing MHC expression in APCs, which in turn alters T cell metabolism, promoting a Warburg-like effect.

### Materials and Methods

### Study Design

Frozen splenocytes and PBLs collected at the time of termination were use from Cohorts (B, C and D) of previously published study (A. Singh, S. Ramachandran, M. L. Graham, S. Daneshmandi, D. Heller, W. L. Suarez-Pinzon, A. N. Balamurugan, J. D. Ansite, J. J. Wilhelm, A. Yang, Y. Zhang, N. P. Palani, J. E. Abrahante, C. Burlak, S. D. Miller, X. Luo, B. J. Hering, Long-term tolerance of islet allografts in nonhuman primates induced by apoptotic donor leukocytes. Nat Commun 10, 3495 (2019)) included purpose-bred monkey (Macaca mulatta) recipients of Indian origin obtained from the National Institute of Health and Infectious Diseases colony at AlphaGenesis, Inc, Yemassee, SC. For full clinical and demographic information of the study Cohort please referred to our previous report. To enable baseline comparison, we included naïve controls (Cohort N; n=3 males, 4.3±2.1 years, 6.2±1.6 kg). Naïve controls (Cohort N) consisted of male NHPs previously characterized prior to any transplant-related interventions and maintained under the same conditions as the experimental groups. The tolerant (C) and nontolerant (B and D) Cohorts included both sexes. Cohort B consisted of one-DRB matched islet transplant recipients treated with induction immunosuppression only and had graft survival less than 203 days. Cohort C consisted of one-DRB matched islet transplant recipients treated with peritransplant infusions of ADLs along with induction immunosuppression and had graft survival >365 days. Cohort D consisted of completely mismatched islet transplant recipients treated with peritransplant infusions of ADLs along with induction immunosuppression and had graft survival <150 days.

Sex as a biological variable. NHPs of both sexes were used in the study, and no significant difference was observed. Tolerant (Cohort C) and nontolerant (Cohorts B and D) animals included both sexes, prior studies in this model have not reported sex-based differences in splenic CD4⁺ T cell, Tr1, or PD-1⁺ Tex phenotypes at baseline.

### Sample Thawing, Preparation and Masscytometry Staining

Cryopreserved splenocytes and PBLs from allo-islet transplant NHPs (Cohorts B: n=4, C: n=3, D: n=2) and control naïve NHPs (Cohort N: n=3) were thawed, rested for 1 hour, and then analyzed using a tetramer-integrated CyTOF panel. Antibodies were either conjugated in-house using MaxPar X8 or MCP9 polymer kits (Standard BioTools) or purchased pre-conjugated. We employed a validated CyTOF panel, with MHC class II tetramers-PE, loaded with mismatched donor MHC I allopeptide to monitor the dynamics and heterogeneity of allospecific CD4⁺ T cells. Details on tissue specimens and antibody panels are provided in Tables 3-5. Cell viability was assessed using 5 μM cisplatin (Standard BioTools). After blocking nonspecific staining with human Fc block (BD Biosciences), cells were labeled with surface antibodies and recipient-specific FITC, PE, and APC-conjugated tetramers (0.5 or 1 μgmL-1 HLA class II tetramer) for 30 minutes at 4° C. Following a wash with MaxPar Cell Staining Buffer (CSB), cells were labeled with anti-FITC (160Gd), PE (165Ho), and APC (163Dy) metal tag antibodies for 30 minutes at 4° C. After surface staining, cells were fixed, permeabilized, and stained with intracellular antibody cocktails for 30 minutes at 4° C., then incubated overnight in 125 nM Intercalator-Ir (Standard BioTools) at 4° C.

CyTOF data acquisition. Prior to acquisition, samples were washed, counted, diluted to 0.5 million cells/mL with 10% calibration beads, filtered, and acquired at under 500 events after extensive preparation.

Tetramer preparation for CyTOF system: To track and analyze allospecific CD4⁺ T cells, we implemented a methodological approach involving the utilization of a customized 43-antibody CyTOF panel with validated specificities for macaque T cell surface and intracellular antigens (Table 4). This panel was integrated with MHC class II tetramers-PE, specifically loaded with mismatched donor MHC I allopeptide. In our procedure, we identified peptides from Mamu MHC class I that exhibited high binding affinity for HLA DRB113 or HLA DRB114 using the Immune Epitope Database Analysis resource. Synthetic peptides obtained from Genscript USA Inc. were loaded onto HLA DRB113 or HLA DRB114 tetramers. These peptides tetramers underwent rigorous testing and screening for flow cytometry application. Further, we performed optimization steps to ensure their suitability for application in the CyTOF system, thereby ensuring the accuracy and reliability of our tetramer-based approach.

### CyTOF Analysis Workflow

After acquisition, data signal from each sample were normalized using EQ Four Element Calibration Beads (EQ Beads, 201078, Standard BioTools), according to manufacturer's instructions. The generated fcs files were then cleaned based on beads, Barium/Cesium contamination, doublet, and dead cell removal (Cisplatin^{low}) and cells populations were manually gated using FlowJo software V10.8.1 (TreeStar) to keep only DNA1⁺CD45⁺CD3⁺CD4⁺ cells events for subsequent clustering and high-dimensional analyses. To determining the distribution and abundance of major CD4⁺ T cell subsets we carefully selected allospecific (II-Tet^{pMHC-I+}) total CD4⁺ T cell, Tr1 cells, exhausted CD4⁺ T cells, using their canonical markers. Individual fcs file were generated for selected allospecific-tetramer (II-Tet^{pMHC-I+}) CD4⁺ T cells, allospecific-tetramer (II-Tet^{pMHC-I+}) LAG-3⁺CD49b⁺/CD4⁺ Tr1 cells and allospecific-tetramer (II-Tet^{pMHC-I+}) PD-1^{hi}CD4⁺ Tex cells events for subsequent clustering and high-dimensional analyses using R package. Individual FCS files were imported into R (v4.1.0), counts data were arcsin transformed, and the counts data were converted to Seurat (4.3.0) data objects followed by normalization and scaling with the SCTransform algorithm implemented in Seurat. For each cell-type subset enumerated above, we integrated individual samples using the anchor-based integration method in Seurat. Following integration, we performed PCA, created a shared-nearest-neighbor network based on the cells PCA embeddings and inferred clusters across 12 clustering resolutions (0.1-1.2). Subsequent visualizations were generated after dimension reduction via Uniform Manifold Approximation and Projection (UMAP).

### Clustree Visualization

The clustree R package was used to determine the appropriate resolution for clustering subsets of CD4⁺ T cell at which the clusters appear most stable and meaningful. Clustering was performed on total CD4⁺ T cells and on each individual CD4⁺ T cell subsets files (allospecific Tex cells, Tregs and Tr1 cells) to determine the resolution that accurately captures the heterogeneity of each subset while avoiding over-splitting or under-clustering. This approach enhances the reliability and interpretability of our clustering results. Table 6 shows the define resolution and associate number of clusters use for each subset of CD4⁺ T cells.

### Trajectory Analysis and CyTOF Pseudotime

Following cluster inference, we inferred developmental trajectories in each cell-type subset using slingshot (v2.0.0). Briefly, Seurat objects were converted to single-cell experiment (see) objects. We specified the following populations as starting points for trajectory inference (c1: TEMRA; CD95⁺, CCR7⁻CD45RA⁺), memory Teff, Tr1 and Tex cell differentiation. We generated temporally smoothed counts values across trajectories after fitting a general additive model (GAM) to describe the relationship between expression and pseudotime. These counts were then visualized as line plots and heatmaps to show expression changes over time.

### Cell-Chat; Cell-Cell Communication

To infer cell-cell communication, CellChat uses a manually curated database of receptor-ligand interactions for mouse and human, the latter of which we used in this study. To do so, we simply converted our CyTOF markers to their human gene ortholog names. We then converted our Seurat object to a CellChat object and subset down to signaling genes, from which we identified over-expressed features. From the over-expressed features, we inferred over-expressed interactions and identified significant interactions by calculating the probability of communication which involves a calculation of the average expression per cluster, for which we used the ‘trimean’ method. From the inferred interactions, we also calculated the pathway-level communication probability and inferred the total communication network. (world-wide-web at github.com/aherman-umn/SDI).

### Donor-Specific T Cells and Intracellular Cytokines

To assess donor-specific polyfunctional CD4 T cell responses in tolerant (Cohort C) and non-tolerant (Cohort B) NHPs, splenocytes were thawed, rested, and exposed to donor cells for 24 hours. In the final 5 hours, cells were stimulated with cell stimulation cocktails (1:500× dilution) in the presence of Brefeldin A. Intracellular cytokines (IC), Granzyme, and Perforin were then assessed via flow cytometry. Cells were stained for extracellular markers, followed by fixation/permeabilization and IC staining. Data from 100,000 events were analyzed using FlowJo, with background cytokine responses subtracted from stimulated samples.

Cell Culture, In Vitro Tr1 Generation, Tr1 Cell Sorting, and MLR with siRNA-Mediated Gene Silencing

PBLs were isolated from donor and recipient NHPs using density gradient centrifugation. To induce donor-specific Tr1 cell differentiation, ADLs were generated by treating donor B cells with ethylene carbodiimide (ECDI) and co-culturing them with recipient PBLs. Cultured cells were phenotypically characterized by staining with fluorescently conjugated antibodies and sorted via flow cytometry based on LAG-3, CD49b, and CD4 expression, while excluding debris and dead cells. The identified Tr1 cells exhibited a CD49b⁺ LAG-3⁺ CD4⁺ phenotype and expressed high levels of Areg and EGFR. FACS sorting was performed with an FACS Melody (BD Biosciences). Sorted Tr1 cells were rested overnight in culture medium and transfected with Areg-specific siRNA (1 μM) using appropriate delivery reagents to knock down Areg expression along with scrambled siRNA controls (Areg⁺ scmbl) as per the manufacturer's instructions (Dharmacon™, GE Healthcare). The knockdown efficiency was assessed via quantitative PCR and flow cytometry.

To evaluate the functional role of Areg Tr1 cells, T cell-enriched responder (respT) cells were isolated from recipient PBLs and stimulated with irradiated donor cells. These Tres cells were co-cultured for six days in the presence or absence of Areg-siRNA-treated (Areg⁻ siRNA) vs. control (Areg⁺ scmbl) Tr1 cells. In a parallel experiment, Nur77-specific siRNA (1 μM) was used to silence Nur77 expression in Tres cells (Nur77⁻ siRNA), with scrambled siRNA controls (Nur77⁺ scmbl), included according to the manufacturer's instructions. Functional assessment of Tres cells under different treatment conditions was performed using: 1-Multicolor flow cytometry to measure donor-specific T cell proliferation and epigenetic modifications (acetylation). 2-Seahorse extracellular flux analysis to evaluate cellular metabolism and 3-Quantitative RT-PCR to analyze metabolic gene expression.

### Quantitative RT-PCR

Total RNA was extracted from cells using the QIAGEN RNeasy Mini Kit, and cDNA synthesis was performed with 1 μg of RNA using the iScript cDNA Synthesis Kit (Bio-Rad). Gene expression was analyzed via SYBR Green-based qPCR (Bio-Rad) using primers listed in Table 12. β-Actin was used as an internal control to normalize mRNA levels and ensure accurate gene expression analysis.

TABLE 12
Metabolic genes, primers and sequences

| Genes | Primers | Sequences |
|---|---|---|
| PFK1 | Forward | 5′ ATTCGGGCTGTGTTCTGG 3′ |
| | Reverse | 5′ TGGCTAGGATTTTGAGGATGG 3′ |
| PKLR1 | Forward | 5′ GAGTCTTCCCCTTGCTTTACC 3′ |
| | Reverse | 5′ GAGCTTTCCACTTTCAATGCC 3′ |
| IDH1 | Forward | 5′ AATATTGTGGGTGGCACGG 3′ |
| | Reverse | 5′ AGTTGCTCTGTATTGATCCCC 3′ |
| AKD | Forward | 5′ GACCAAAGCCGAACAGTTTTAC 3′ |
| | Reverse | 5′ ATGAGTTGTGTAGGATGGCAG 3′ |
| CS | Forward | 5′ TGAGGGTGGCAATGTAAGTG 3′ |
| | Reverse | 5′ TTAGCCAGACAAGCACTTCC 3′ |

Sequences are SEQ ID Nos: 24-33 in order of appearance in Table 12.
Seahorse Mito Stress Assay for Metabolic Profiling of respT Cells

Oxygen consumption rate (OCR) was measured using the Seahorse XFe96 Bioanalyzer (Agilent) in Tres cells (Nur77⁺ scmbl vs Nur77⁻ siRNA) cultured with Tr1 cells (Areg⁻ siRNA vs Areg⁺ scmbl) for six days. Before plating, cells were washed in XF Base Media (Agilent) supplemented with 10 mM glucose, 1 mM sodium pyruvate, and 2 mM L-glutamine (Gibco), adjusted to pH 7.4 at 37° C. Cells (2×10⁵ per well) in triplicate were then plated onto Poly-D-Lysine (50 μg/mL) (Sigma)-coated Seahorse culture plates.

After adherence and equilibration, OCR and ECAR were recorded using the Seahorse Mito Stress Assay (Agilent), following the sequential injection of oligomycin (1 μM), FCCP (1.5 μM), and a mix of antimycin A and rotenone (0.5 μM each). The assay parameters included 3 min mixing, no wait time, and 3 min measurement, repeated 3-4 times at baseline and after each compound addition.

Statistics. Differential abundance of cell-clusters was determined using edgeR as described in the Bioconductor book “Multi-Sample Single-Cell Analyses with Bioconductor” (on the world-wide-web at bioconductor.org/books/3.13/OSCA.multisample/).

### Statistical Analysis

Statistical analysis of mass-cytometric data distributions of values within all Cohorts were checked for Gaussian distribution. Owing to the small sample size and non-normal distribution, we used the non-parametric Mann-Whitney U test in GraphPad Prism (v9) followed by post hoc analysis with the Holm-Sidak method for comparisons between two groups and unpaired t test. For all statistical tests, *P<0.05 and **P<0.005.

### Example 2

### Dissecting the Phenotype of Allospecific CD4⁺ T Cell in Nonhuman Primates with Long-Term Tolerance of Islet Allograft Display Distinct Exhaust and Regulatory Immune Signatures

Background: For islet cell replacement strategies to be leveraged to their full potential in diabetes care, rejection of transplanted human primary or stem cell-derived islets must be prevented without maintenance immunosuppression. Two peritransplant infusions of apoptotic donor leukocytes (ADLs) under induction immunosuppression (IIS) safely induced long-term tolerance via depletion of alloreactive T cells and expansion of several regulatory cell subsets. A deeper understanding of the immune mechanisms and signatures associated with tolerance induced by the ADL+IIS regimen improves the likelihood of successful clinical translation in islet cell transplantation.

Methods: A 45-parameter CyTOF panel was employed, which spans cell surface and intracellular antigen, validated for use in nonhuman primates, to analyze the identity, abundance, and phenotypic profile of circulatory and splenic T cell subsets in rhesus macaques undergoing islet transplantation by cohort: A (1 DRB-matched with IIS only), B (1 DRBmatched with ADLs+IIS), and C (complete MHC mismatch with ADLs+IIS), each with an n=3. To track allospecific CD4+ T cells, we incorporated MHC class II tetramers that were loaded with mismatched donor MHC class I peptides into the CyTOF panel. The bioinformatics analysis was by unsupervised UMAP clustering to investigate the heterogeneity and functional profiles of T cell subsets.

Results: The analysis demonstrated the ADL+IIS protocol induced exhaustion in allospecific CD4+ T cells. The presence of nine distinct clusters of allospecific MHC class I+CD4+ T cells were identified. Among these clusters, four were T cells expressing markers associated with exhaustion (C0, C1, C3, and C4). The clusters C0 and C1 (abundant in blood) were expanded in cohort C, while clusters C3 and C4 (abundant in spleen) showed comparable frequencies across cohorts. Next, the regulatory heterogeneity of MHCI+ regulatory T cells (Tregs) was examined, and nine distinct clusters were identified, with four of nine clusters recognized as heterogenous subpopulation of activated allospecific regulatory T cells, either showing the phenotype of activated FoxP3+ Tres (C5, C6, and C7) or LAG-3+ CD49bhi FoxP3low Tregs (C8). Among these regulatory clusters, C5 and C7 dominated in the peripheral circulation while C6 and C8 showed high abundance in the spleen compartment. ADL+IIS promotes expansion of allospecific activated and functional regulatory T cells in islets Ix tolerant RM.

Conclusions: The preclinical data in nonhuman primates suggests that the ADLs+IIS regimen in 1 DRB-matched islet transplant recipients promotes both exhaustion of several T cell subsets and activation of distinct Treg and Tr1 regulatory cell subsets.

### Example 3

### Assay Development & Kit Commercialization

The target cells are T regulatory (Treg) cells having marker
CCR2^{hi}TIGIT^{hi} and/or Helios^{hi}USP22^{hi} and/or
c6; CCR2^{hi}TIGIT^{hi}Areg⁺ (>1.5-fold higher in the peripheral blood of Tolerant vs nontolerant)
and/or
c8; Helios^{hi}USP22^{hi} (>1.5-fold higher in the peripheral blood of Tolerant vs nontolerant)

In certain embodiments, the post-procedure frequency is at least 1.5-fold greater than the baseline frequency. In certain embodiments, the frequency is at least 1.5, 2, 3, 4, 5, 6, 7, 8, 9, 10, 20, 30, 40, 50, 60, 70, 80, 90, or 100-fold greater than the baseline frequency.

In one embodiment, the target cells are T regulatory (Treg) cells having marker
CCR2^{hi}TIGIT^{hi} and/or Helios^{hi}USP22^{hi} and/or
c6; CCR2^{hi}TIGIT^{hi}Areg⁺ (>25-fold higher in the peripheral blood of Tolerant vs nontolerant)
and/or
c8; Helios^{hi}USP22^{hi} (>4-fold higher in the peripheral blood of Tolerant vs nontolerant)

The target cells are T regulatory Type 1 (Tr1) cells having one or more combination of markers
PD-1^{hi}CD69^{hi}Helios^{hi} and/or
TIGIT^{hi} CCR2^{hi}ST2^{hi}GrnzB⁺ FOXP3−
c4: Areg^{hi} TIGIT^{hi} CCR2^{hi} GrnzB⁺ ST2^{hi} FOXP3− Tr1 cells
(>1.5-fold higher in the peripheral blood of tolerant vs nontolerant)

In certain embodiments, the post-procedure frequency is at least 1.5-fold greater than the baseline frequency. In certain embodiments, the frequency is at least 1.5, 2, 3, 4, 5, 6, 7, 8, 9, 10, 20, 30, 40, 50, 60, 70, 80, 90, or 100-fold greater than the baseline frequency.

In one embodiment, the target cells are T regulatory Type 1 (Tr1) cells having one or more combination of markers
PD-1^{hi}CD69^{hi}Helios^{hi} and/or
TIGIT^{hi} CCR2^{hi}ST2^{hi} GrnzB⁺ FOXP3−
c4: Areg^{hi} TIGIT^{hi} CCR2^{hi} GrnzB⁺ ST2^{hi} FOXP3− Tr1 cells
(>10-fold higher in the peripheral blood of tolerant vs nontolerant)

In one embodiment, the target cells are exhaust CD4 T cells (Tex) having marker
c1: PD-1⁺EOMES⁺CD127^{hi} Helios⁺TOX⁺TCF-1⁻
(>1.5-fold higher in the peripheral blood and >1.5-fold higher in spleen of tolerant vs nontolerant)

In certain embodiments, the post-procedure frequency is at least 1.5-fold greater than the baseline frequency. In certain embodiments, the frequency is at least 1.5, 2, 3, 4, 5, 6, 7, 8, 9, 10, 20, 30, 40, 50, 60, 70, 80, 90, or 100-fold greater than the baseline frequency.

The target cells are exhaust CD4⁺ T cells (Tex) having marker
c1: PD-1⁺EOMES⁺CD127^{hi} Helios⁺TOX⁺TCF-1⁻
(>2-fold higher in the peripheral blood and >11-fold higher in spleen of tolerant vs nontolerant)

### 1. Flow Cytometry Kit (RUO→Laboratory Development Test)

1. 12-color dried antibody cocktail with lyophilized QC controls.
2. Single-tube format, compatible with BD/FACS and Cytek Aurora.

Includes preloaded gating templates for Treg, Tr1, Tex Tol modules. (Table1)

### (I) Final≤12-Marker Core List (Flow Cytometry Version); Surface+Intracellular, Whole Blood Panel

1. CD4
2. CD127
3. FOXP3 (i.c.)
4. Helios/USP22 (→Treg stability, choose one)
5. Areg (Areg-EGFR axis→Tr1 stability)
6. CD49b
7. LAG-3
8. ST2
9. CCR2
10. PD-1
11. TOX/EOMES (i.c.)
12. TIGIT (Tex co-inhibitor)

### (II) Tolerance Consolidation Index (TCI)—Draft Scoring Sheet

| Module | Formula | Clinical Interpretation | Value (z-score |
|---|---|---|---|
| Treg module | %CD4⁺FoxP3⁺CD127^{low} × USP22/Helios, CCR2 MFI | ≥3-fold, High score = stable regulatory | 1 |
| | | compartment | |
| Tr1 module | %CD4⁺FOXP3⁻ CD49b⁺LAG-3⁺ × AREG expression | ≥3-fold, Higher = tolerance consolidation | 1 |
| Tex module | %CD4⁺PD-1⁺CD127^{hi} TIGIT⁺TOX⁺ (±EOMES) vs | Higher = exhaustion low risk of rejection | 1 |
| | TIGIT⁻TOX⁻ PD-1⁺/CD4 (activated T cell) | Ratio ≥1:1 | |
| Composite TCI | z(Reg + Tr1) + z(Tex) | Green ≥ +3, Yellow <3-2, Red <0 | +3 |

### Interpretation (Research Use Only)

High TCI suggests consolidated tolerance signature.
Recommendation: repeat in 4-6 weeks to confirm stability.

In certain embodiments, the present invention provides kit comprising a panel of binding reagents, wherein the reagents individually specific for CD4, CD127, FOXP3 (i.c.), Areg, CD49b, LAG-3, ST2, CCR2, PD-1, TOX/EOMES (i.c.), TIGIT, and Helios and/or USP22.

In certain embodiments, the binding reagents are antibodies.

In certain embodiments, the kit is a single-tube format.

In certain embodiments, the kit is compatible with BD/FACS and Cytek Aurora.

In certain embodiments, the kit further comprising preloaded gating templates for Treg, Tr1, Tex Tol modules.

In certain embodiments, the preloaded gating templates for the Treg module comprises % CD4⁺FoxP3⁺CD127low×USP22/Helios, CCR2 MFI.

In certain embodiments, the preloaded gating templates for the Tr1, module comprises % CD4⁺FOXP3⁻ CD49b⁺LAG-3⁺×AREG expression.

In certain embodiments, the preloaded gating templates for the Tex Tol module % CD4⁺PD-1⁺CD127hi TIGIT⁺TOX⁺ (±EOMES) vs TIGIT-TOX-PD-1+/CD4 (activated T cell).

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of identifying a transplant recipient patient having transplantation tolerance induced by donor antigen administered under cover of transient immunotherapy, comprising:
(a) assaying a first blood sample from the patient to detect a baseline frequency of target cells, wherein the first blood sample is obtained pre-tolerization, pre-transplant, and pre-initiation of transient immunotherapy,
(b) assaying a second blood sample from the patient to detect a post-procedure frequency of target cells, wherein the second sample is obtained post-tolerization, post-transplant, and post-initiation of transient immunotherapy; and
(c) identifying the patient as having transplantation tolerance/immune acceptance induced by the donor antigen when the post-procedure frequency is at least 1.5-fold greater than the baseline frequency,
wherein the target cells are T regulatory (Treg) cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻FOXP3⁺; and/or
wherein the target cells are T regulatory Type 1 (Tr1) cells having one or more combination of markers PD-1^{hi}CD69^{hi}Helios^{hi}, TIGIT^{hi} CCR2^{hi}ST2^{hi} GrzB⁺FOXP3⁻, Areg^{hi} TIGIT^{hi} CCR2^{hi} GrnzB⁺ ST2^{hi} FOXP3⁻ and/or CD4⁺CD49b⁺LAG3⁺; and/or
wherein the target cells are exhaust CD4 T cells (Tex) having markers PD-1⁺EOMES⁺ CD127^{hi} Helios⁺TOX⁺TCF-1⁻.

2. A method, comprising:
(a) obtaining a first blood sample from a transplant recipient patient to detect a baseline frequency of target cells, wherein the first blood sample is obtained pre-tolerization, pre-transplant, and pre-initiation of transient immunotherapy,
(b) obtaining a second blood sample from the patient to detect a post-procedure frequency of target cells, wherein the second sample is obtained post-tolerization, post-transplant, and post-initiation of transient immunotherapy,
(c) assaying the first and second blood samples to detect levels of target cells before and after tolerization, and
(d) identifying the transplant recipient patient as having transplantation tolerance/immune acceptance induced by donor antigens infused under cover of transient immunotherapy when the post-procedure frequency is at least 1.5-fold greater than the baseline frequency,
wherein the target cells are T regulatory (Treg) cells having one or more combination of markers Helios^{hi}USP22^{hi}, CCR2^{hi}TIGIT^{hi}, CCR2^{hi}TIGIT^{hi}Areg⁺, and/or CD4⁺CD25⁺CD127⁻FOXP3⁺; and/or
wherein the target cells are T regulatory Type 1 (Tr1) cells having one or more combination of markers PD-1^{hi}CD69^{hi}Helios^{hi}, TIGIT^{hi} CCR2^{hi}ST2^{hi}GrnzB⁺FOXP3⁻, Areg^{hi} TIGIT^{hi} CCR2^{hi} GrnzB⁺ST2^{hi} FOXP3⁻ and/or CD4⁺CD49b⁺LAG3⁺; and/or
wherein the target cells are exhaust CD4 T cells (Tex) having one or more combination of markers PD-1⁺EOMES⁺ CD127^{hi} Helios⁺TOX⁺TCF-1⁻,
wherein the identification is with a multiparameter single flowcytometry panel comprising 12 binding reagents that specifically recognize CD4, FOXP3, CD49b, LAG-3, PD-1, Helios/USP22, CCR2, ST2, TIGIT, TOX/EOMES, CD127, and Areg to determine these regulatory and exhaust subsets.

3. The method of claim 1, wherein the donor antigens are apoptotic donor leukocytes (ADLs), donor-specific transfusion (DST) nanoparticles conjugated with donor peptides or encapsulating donor peptides, and/or apoptotic recipient leukocytes conjugated with donor peptides.

4. A method of treating a transplant recipient patient, the method comprising:
(a) identifying the transplant recipient patient as using the method of claim 1, and
(b) treating the transplant recipient patient by ceasing to administer immunosuppressants.

5. The method of claim 4, wherein the Tr1 cells exhibit indirect specificity for at least one mismatched donor MHC class I peptide.

6. The method of claim 1, wherein the target cells are blood cells.

7. The method of claim 1, wherein the patient has received two peritransplant, intravenous infusions of apoptotic donor leukocytes.

8. The method of claim 1, wherein the transient immunotherapy comprises at least one immunosuppressant.

9. The method of claim 8, wherein the immunosuppressant is an inhibitor of CD40:CD40L co-stimulation, an mTOR inhibitor, and concomitant anti-inflammatory therapy targeting proinflammatory cytokines.

10. The method of claim 1, wherein the transient immunotherapy comprises an anti-inflammatory agent.

11. The method of claim 1, wherein the transplant is an allotransplant.

12. A kit comprising a panel of binding reagents, wherein the reagents individually specific for CD4, CD127, FOXP3 (i.c.), Areg, CD49b, LAG-3, ST2, CCR2, PD-1, TOX/EOMES (i.c.), TIGIT, and Helios and/or USP22.

13. The kit of claim 12, wherein the binding reagents are antibodies.

14. The kit of claim 12, wherein the kit is a single-tube format.

15. The kit of claim 14, wherein the kit is compatible with BD/FACS and Cytek Aurora.

16. The kit of claim 14, further comprising preloaded gating templates for Treg, Tr1, Tex Tol modules.

17. The kit of claim 16, wherein the preloaded gating templates for the Treg module comprises % CD4⁺FoxP3⁺CD127low×USP22/Helios, CCR2 MFI.

18. The kit of claim 16, wherein the preloaded gating templates for the Tr1, module comprises % CD4⁺FOXP3⁻CD49b⁺LAG-3⁺×AREG expression.

19. The kit of claim 16, wherein the preloaded gating templates for the Tex Tol module % CD4⁺PD-1⁺CD127^{hi} TIGIT⁺TOX⁺ (±EOMES) vs TIGIT-TOX-PD-1+/CD4 (activated T cell).
